SEQUENCE LISTING (9 sequences)

SEQ ID NO: 1 - DNA, 21 bp, Synthetic construct
  source 1..21: mol_type = other DNA
taccattgagttgtgcgtggg

SEQ ID NO: 2 - DNA, 23 bp, synthetic construct
  source 1..23: mol_type = other DNA
gccattgtttcgtccataggaga

SEQ ID NO: 3 - DNA, 21 bp, synthetic construct
  source 1..21: mol_type = other DNA
gggagaccttggctgtaatgg

SEQ ID NO: 4 - DNA, 21 bp, synthetic construct
  source 1..21: mol_type = other DNA
ttcggttcccacatccagaat

SEQ ID NO: 5 - DNA, 24 bp, synthetic construct
  source 1..24: mol_type = other DNA
gtactcttgtccatctgttctctg

SEQ ID NO: 6 - DNA, 21 bp, synthetic construct
  source 1..21: mol_type = other DNA
ccattcccgagctattctgtt

SEQ ID NO: 7 - RNA, 3338 nt, Homo sapiens
  source 1..3338: mol_type = mRNA
tgctgcagtcagcaccgtcaccccagcagcatccgccgcctgcaccgcgcgtgcggcccgccccggcctgaccccgccgccgaacccggcgccagccatggagcccgaagccccccgtcgccgccacacccatcagcgcggctacctgctgacacggaaccctcacctcaacaaggacttggcctttaccctggaagagagacagcaattgaacattcatggattgttgccaccttccttcaacagtcaggagatccaggttcttagagtagtaaaaaatttcgagcatctgaactctgactttgacaggtatcttctcttaatggatctccaagatagaaatgaaaaactcttttatagagtgctgacatctgacattgagaaattcatgcctattgtttatactcccactgtgggtctggcttgccaacaatatagtttggtgtttcggaagccaagaggtctctttattactatccacgatcgagggcatattgcttcagttctcaatgcatggccagaagatgtcatcaaggccattgtggtgactgatggagagcgtattcttggcttgggagaccttggctgtaatggaatgggcatccctgtgggtaaattggctctatatacagcttgcggagggatgaatcctcaagaatgtctgcctgtcattctggatgtgggaaccgaaaatgaggagttacttaaagatccactctacattggactacggcagagaagagtaagaggttctgaatatgatgattttttggacgaattcatggaggcagtttcttccaagtatggcatgaattgccttattcagtttgaagattttgccaatgtgaatgcatttcgtctcctgaacaagtatcgaaaccagtattgcacattcaatgatgatattcaaggaacagcatctgttgcagttgcaggtctccttgcagctcttcgaataaccaagaacaaactgtctgatcaaacaatactattccaaggagctggagaggctgccctagggattgcacacctgattgtgatggccttggaaaaagaaggtttaccaaaagagaaagccatcaaaaagatatggctggttgattcaaaaggattaatagttaagggacgtgcttccttaacacaagagaaagagaagtttgcccatgaacatgaagaaatgaagaacctagaagccattgttcaagaaataaaaccaactgccctcataggagttgctgcaattggtggtgcattctcagaacaaattctcaaagatatggctgccttcaatgaacggcctattatttttgctttgagtaatccaactagcaaagcagaatgttctgcagagcagtgctacaaaataaccaagggacgtgcaatttttgccagtggcagtccttttgatccagtcactcttccaaatggacagaccctatatcctggccaaggcaacaattcctatgtgttccctggagttgctcttggtgttgtggcgtgtggattgaggcagatcacagataatattttcctcactactgctgaggttatagctcagcaagtgtcagataaacacttggaagagggtcggctttatcctcctttgaataccattagagatgtttctctgaaaattgcagaaaagattgtgaaagatgcataccaagaaaagacagccacagtttatcctgaaccgcaaaacaaagaagcatttgtccgctcccagatgtatagtactgattatgaccagattctacctgattgttattcttggcctgaagaggtgcagaaaatacagaccaaagttgaccagtaggataatagcaaacatttctaactctattaatgaggtctttaaacctttcataatttttaaaggttggaatcttttataatgattcataagacacttagattaagattttactttaacagtctaaaaattgatagaagaatatcgatataaattgggataaacatcacatgagacaattttgcttcactttgccttctggttatttatggtttctgtctgaattattctgcctacgttctctttaaaagctgttgtacgtactacggagaaactcatcatttttatacaggacactaatgggaagaccaaaattactaataaattgacataaccaacattaaaactcataattattttgttgaccattttgttaaaatctacttttcaaaaaaaaaaagctagaaatgaatctaggcgtaggtgaacttttgctaagcagaaataacactactttgttgcctagagaaagataacttctcaagtatttttattccagtcctagatcatatatgttcttttgtgcaacggaattctaacagttctaagagaaagatcactgctgtttacagcgccttgtgcagccttagattttaatattcttttgtcattgttacatctcatagagtaaagctcttattaccttgatcctgagtcagaaatcccacctgaaatcaccttttttcccccttgatcaaacatcccatccttcagctaccatactgttgctacagggattttgtggactgtggcccctgtcccgaggttggcaccttcagttcagcacagcctgagcagtgagaaggtctgaaaggagagtatatagttaagatccttgagaaagggctgcctgaggaactgacctcttaaagatctcaggatctttaagacaacaagttaggttcctactggagttacctgccagaatggcctcttaattaactcaggtaatgaagagctaactgtgttataatcatcttgcttttgcctgaatttggagaaagtattataattaagttcccagtatcagaaatgtccttacataagattaaaatatcttgatgactaataccattctatgagaaagagtagttatatgcccagactgtattaatttactttagaaactaatgtttgaagtaatggaaaaaattttaaattataaagctaaggtgcaataacatttgctacttatttatagaattatttgaagaattttgtttttgaagtaatgctttaaggagtataagatattcaagataaattatactataaaatgattttattgaaagttgaaggttacacaaattgttttaggtatgagcagaagaggttaaggtatttctaaaggtaacatatagtcaagagtttcctcaaaatagttatttggagaagaatcagaatgtctgtgtatttcttgtctgtttctatgttgtcttatagctctgactaaatgtgtttacctatgcaaaagatttattaaagcatagaaaaggtgaatgaataaaaatataaaataattgtc

SEQ ID NO: 8 - RNA, 1720 nt, Homo sapiens
  source 1..1720: mol_type = mRNA
atggagcccgaagccccccgtcgccgccacacccatcagcgcggctacctgctgacacggaaccctcacctcaacaaggacttggcctttaccctggaagagagacagcaattgaacattcatggattgttgccaccttccttcaacagtcaggagatccaggttcttagagtagtaaaaaatttcgagcatctgaactctgactttgacaggtatcttctcttaatggatctccaagatagaaatgaaaaactcttttatagagtgctgacatctgacattgagaaattcatgcctattgtttatactcccactgtgggtctggcttgccaacaatatagtttggtgtttcggaagccaagaggtctctttattactatccacgatcgagggcatattgcttcagttctcaatgcatggccagaagatgtcatcaaggccattgtggtgactgatggagagcgtattcttggcttgggagaccttggctgtaatggaatgggcatccctgtgggtaaattggctctatatacagcttgcggagggatgaatcctcaagaatgtctgcctgtcattctggatgtgggaaccgaaaatgaggagttacttaaagatccactctacattggactacggcagagaagagtaagaggttctgaatatgatgattttttggacgaattcatggaggcagtttcttccaagtatggcatgaattgccttattcagtttgaagattttgccaatgtgaatgcatttcgtctcctgaacaagtatcgaaaccagtattgcacattcaatgatgatattcaaggaacagcatctgttgcagttgcaggtctccttgcagctcttcgaataaccaagaacaaactgtctgatcaaacaatactattccaaggagctggagaggctgccctagggattgcacacctgattgtgatggccttggaaaaagaaggtttaccaaaagagaaagccatcaaaaagatatggctggttgattcaaaaggattaatagttaagggacgtgcttccttaacacaagagaaagagaagtttgcccatgaacatgaagaaatgaagaacctagaagccattgttcaagaaataaaaccaactgccctcataggagttgctgcaattggtggtgcattctcagaacaaattctcaaagatatggctgccttcaatgaacggcctattatttttgctttgagtaatccaactagcaaagcagaatgttctgcagagcagtgctacaaaataaccaagggacgtgcaatttttgccagtggcagtccttttgatccagtcactcttccaaatggacagaccctatatcctggccaaggcaacaattcctatgtgttccctggagttgctcttggtgttgtggcgtgtggattgaggcagatcacagataatattttcctcactactgctgaggttatagctcagcaagtgtcagataaacacttggaagagggtcggctttatcctcctttgaataccattagagatgtttctctgaaaattgcagaaaagattgtgaaagatgcataccaagaaaagacagccacagtttatcctgaaccgcaaaacaaagaagcatttgtccgctcccagatgtatagtactgattatgaccagattctacctgattgttattcttggcctgaagaggtgcagaaaatacagaccaaagttgaccagtagg

SEQ ID NO: 9 - protein, 572 aa, Homo sapiens
MEPEAPRRRHTHQRGYLLTRNPHLNKDLAFTLEERQQLNIHGLLPPSFNSQEIQVLRVVKNFEHLNSDFDRYLLLMDLQDRNEKLFYRVLTSDIEKFMPIVYTPTVGLACQQYSLVFRKPRGLFITIHDRGHIASVLNAWPEDVIKAIVVTDGERILGLGDLGCNGMGIPVGKLALYTACGGMNPQECLPVILDVGTENEELLKDPLYIGLRQRRVRGSEYDDFLDEFMEAVSSKYGMNCLIQFEDFANVNAFRLLNKYRNQYCTFNDDIQGTASVAVAGLLAALRITKNKLSDQTILFQGAGEAALGIAHLIVMALEKEGLPKEKAIKKIWLVDSKGLIVKGRASLTQEKEKFAHEHEEMKNLEAIVQEIKPTALIGVAAIGGAFSEQILKDMAAFNERPIIFALSNPTSKAECSAEQCYKITKGRAIFASGSPFDPVTLPNGQTLYPGQGNNSYVFPGVALGVVACGLRQITDNIFLTTAEVIAQQVSDKHLEEGRLYPPLNTIRDVSLKIAEKIVKDAYQEKTATVYPEPQNKEAFVRSQMYSTDYDQILPDCYSWPEEVQKIQTKVDQ

(19) United States
(12) Patent Application Publication
(10) Pub. No.: US 2026/0176650 A1
(43) Pub. Date: Jun. 25, 2026

(54) T CELLS WITH INCREASED EXPRESSION OF MALIC ENZYME 1 AND USES THEREOF IN CANCER THERAPY

(71) Applicant: Mayo Foundation for Medical Education and Research, Rochester, MN (US)

(72) Inventors: Haidong Dong, Rochester, MN (US); Joanina K. Gicobi, Rochester, MN (US)

(21) Appl. No.: 19/125,409

(22) PCT Filed: Nov. 3, 2023
(86) PCT No.: PCT/US2023/078597

§ 371 Date: Apr. 29, 2025

Related U.S. Application Data
(60) Provisional application No. 63/422,740, filed on Nov. 4, 2022.

(51) Int. Cl. C12N 15/85

(52) U.S. Cl. CPC C12N 15/85; C07K 2317/76

(57) ABSTRACT

Methods and materials for increasing expression of malic enzyme 1 (ME1) in T cells are provided herein, as are compositions containing ME1 coding sequences or ME1 mRNA, and methods and materials for using T cells with increased ME1 levels to treat mammals having cancer.

### CROSS-REFERENCE TO RELATED APPLICATIONS

This application claims priority from U.S. Provisional Application Ser. No. 63/422,740, filed Nov. 4, 2022. The disclosure of the prior application is considered part of (and is incorporated by reference in) the disclosure of this application.

### SEQUENCE LISTING

This application contains a Sequence Listing that has been submitted electronically as an XML file named “07039-2173WO1.XML.” The XML file, created on Oct. 27, 2023, is 14,545 bytes in size. The material in the XML file is hereby incorporated by reference in its entirety.

### TECHNICAL FIELD

This document relates to methods and materials for increasing expression of malic enzyme 1 (ME1) in T cells, and to methods and materials for using T cells with increased ME1 levels to treat mammals having cancer.

### BACKGROUND

For patients with advanced cancers, effective therapeutic options are limited. Radiation therapy is frequently used to reduce tumor burden and to create opportunity for other therapies, including immunotherapy. Most immunotherapies (e.g., immune checkpoint inhibitors (ICI), vaccines, and T cell therapies) are dependent on the presence of endogenous cytotoxic T lymphocytes (CTLs; also referred to as “cytotoxic T cells”) to be either responsive to ICI or to be a cellular source for T cell transfer therapy. To that end, the functionality of endogenous tumor-reactive CTLs could play a key role in prediction of clinical responses to combination of radiation therapy and immunotherapy. Although CTLs are prone to be exhausted in patients with advanced cancers, some of the CTLs can regain their antitumor activity upon ICI therapy and reject large tumors or metastatic malignances. The term “resilient T cells” (Trs cells) has been proposed to describe the functional state of tumor-reactive cytotoxic T cells that are capable of withstanding tumor burden and recovering quickly from stressful conditions to respond to immunotherapy (Gicobi et al., Cancer Immunol Immunother 69, 2165-2167, 2020; and Gicobi et al., Int J Hematol 2022, doi.org/10.1007/s12185-022-03424-7). Knowledge about to these “rebound” effector T cells is limited, however.

Successful ICI therapy can expand tumor-reactive CD8⁺ T cells with effector phenotype in peripheral blood; these cells may have the potential to replace exhausted T cells inside tumor tissue (An et al., Int J Mol Sci 22, 2021; Sade-Feldman et al., Cell 175, 998-1013.e1020, 2018; Wu et al., Nature 579, 274-278, 2020; Yan et al., JCI Insight 3, 2018, doi.org/10.1172/jci.insight.97828; and Yost et al., Nat Med 25, 1251-1259, 2019). CX3CR1⁺ CD8⁺ T cells that are responsive to ICI therapy in both preclinical and clinical settings have been identified (Yan et al., supra; Yamauchi et al., Nat Commun 12, 1402, 2021; and Zander et al., Immunity 51, 1028-1042.e1024, 2019) and are characterized by a highly cytotoxic state, proliferative activity, and migrative capacity in preclinical models and in the peripheral blood of patients who respond to ICI therapy (Wu et al., supra; Yan et al., supra; and Yamauchi et al., supra). CX3CR1⁺ CD8⁺ T cells may be prototypes of Trs cells in the circulation of patients with advanced cancers.

### SUMMARY

This document is based, at least in part, on the discovery that resilient T cells can explain the presence of highly cytotoxic T cells that are less exhausted and rebound in responses to ICI therapy. This document also is based, at least in part, on the identification of phenotypic and functional characters of resilient T cells, including the discovery that resilient CD8⁺ T cells tend to have low mitochondrial membrane potential (MMP; also referred to as ΔΨm), are highly cytotoxic and express higher levels of malic enzyme 1 (ME1). This document provides compositions containing nucleic acids (e.g., vectors) that include nucleic acid sequences encoding ME1, methods for increasing ME1 levels in cells (e.g., CD8⁺ T cells), and methods for using cells having increased ME1 levels to treat mammals having cancer. For example, methods and materials provided herein can include administering, to human cancer patients, CTLs that express increased levels of ME1.

As demonstrated herein, ICI-therapy responsive CX3CR1⁺ CD8⁺ T cells are endowed with low mitochondrial membrane potential, and the frequency of CX3CR1⁻ CD8⁺ T cells with low mitochondrial membrane potential is increased in patients with metastatic malignances who have better clinical outcomes in responses to ICI therapy and radiation therapy. Further characterization of CD8⁺ T cells with low mitochondrial membrane potential revealed that they are highly cytotoxic and produce less reactive oxygen species (ROS) but express more ME1. Interestingly, overexpression of ME1 can reduce ROS in CD8⁺ T cells and augment tumoricidal activity of CD8⁺ T cells. Importantly, enhanced expression of ME1 in T cells isolated from non-responders improved cytotoxic T cell responses to ICI treatment in vitro. Thus, the studies discussed herein suggested that not all highly cytotoxic CD8⁺ T cells are exhausted, but that some of them are functionally resilient in patients with advanced cancers. As such, modification of ME1 expression in T cells provides a method to avoid T cell exhaustion and to improve the efficacy of cancer immunotherapy.

In general, one aspect of this document features a method for increasing the level of malic enzyme 1 (ME1) in a cell. The method can include, or consist essentially of, introducing into the cell a nucleic acid encoding ME1, and incubating the cell such that the nucleic acid is expressed, thereby increasing the level of ME1 in the cell. The cell can be a T cell. The T cell can be a cytotoxic T lymphocyte (CTL). The CTL can be a CX3CR1⁺ CTL. The T cell can be a chimeric antigen receptor- (CAR-) T cell or a T cell receptor- (TCR-) T cell. The cell can be a human cell. The nucleic acid can be a mRNA. The nucleic acid encoding ME1 can include the nucleotide sequence set forth in SEQ ID NO: 8, or having a nucleotide sequence with at least 90% identity to the nucleotide sequence set forth in SEQ ID NO:8.

In another aspect, this document features a method for treating a mammal. The method can include, or consist essentially of, administering to the mammal a composition containing cells that include an exogenous nucleic acid encoding ME1, such that the cells have an elevated level of ME1. The mammal can be a human. The human can have cancer (e.g., lung cancer, breast cancer, prostate cancer, colorectal cancer, gastric cancer, pancreatic cancer, melanoma, sarcoma, or ovarian cancer). The cells can be peripheral blood mononuclear cells (PBMCs). The cells can be T cells. The T cells can be CTLs. The CTLs can be CX3CR1⁺ CTLs. The cells can have been obtained from the mammal and transfected with the nucleic acid. The T cells can be CAR-T cells or TCR-T cells. The nucleic acid can be a mRNA. The nucleic acid encoding ME1 can include the nucleotide sequence set forth in SEQ ID NO:8, or having a nucleotide sequence with at least 90% identity to the nucleotide sequence set forth in SEQ ID NO:8.

In another aspect, this document features a composition containing PBMCs that contain an exogenous nucleic acid encoding ME1. The nucleic acid can be a mRNA. The nucleic acid can include the nucleotide sequence set forth in SEQ ID NO:8, or a nucleotide sequence with at least 90% identity to the nucleotide sequence set forth in SEQ ID NO:8. The PBMCs can include T cells. The T cells can be CTLs. The CTLs can be CX3CR1⁺ CTLs.

In another aspect, this document features a method for increasing the level of a polypeptide having ME1 activity in a cell. The method can include, or consist essentially of, (a) introducing into the cell a nucleic acid encoding the polypeptide, and (b) incubating the cell such that the nucleic acid is expressed, thereby increasing the level of the polypeptide in the cell. The polypeptide can be a full-length ME1 polypeptide. The polypeptide can be a full-length human ME1 polypeptide. The polypeptide can be a full-length human ME1 polypeptide containing the amino acid sequence set forth in SEQ ID NO: 8. The cell can be a T cell. The T cell can be a CTL. The CTL can be a CX3CR1⁺ CTL. The nucleic acid can be a mRNA.

In still another aspect, this document features a method for treating a mammal. The method can include, or consist essentially of, administering to the mammal a composition containing cells that contain an exogenous nucleic acid that encodes a polypeptide having ME1 activity, wherein the cells have an elevated level of the polypeptide. The polypeptide can be a full-length ME1 polypeptide. The polypeptide can be a full-length human ME1 polypeptide. The polypeptide can be a full-length human ME1 polypeptide containing the amino acid sequence set forth in SEQ ID NO:8. The mammal can be a human. The human can have cancer (e.g., lung cancer, breast cancer, prostate cancer, colorectal cancer, gastric cancer, pancreatic cancer, melanoma, sarcoma, or ovarian cancer). The cells can be PBMCs. The cells can be T cells. The T cells can be CTLs. The CTLs can be CX3CR1⁺ CTLs. The T cells can be CAR-T cells or TCR-T cells. The cells can have been obtained from the mammal and transfected with the nucleic acid. The nucleic acid can be a mRNA.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A shows a representative human ME1 nucleic acid sequence (SEQ ID NO: 7). The coding sequence within SEQ ID NO:7 is underlined (SEQ ID NO:8). FIG. 1B shows a representative human ME1 amino acid sequence (SEQ ID NO:9).

FIGS. 2A-2H show that CX3CR1 and low MMP identify resilient CD8 T cells in patients with advanced cancers upon radiation therapy. FIG. 2A includes graphs plotting the results of flow cytometry analysis for CX3CR1+ with low or high MMP, and their CTL function (degranulation) measured with CD107a expression. FIG. 2B is a diagram of spatially fractionated radiotherapy (SFRT) therapy and the study design used for FIGS. 2C and 2D. FIGS. 2C and 2D include graphs plotting the results of degranulation analyses of CX3CR1⁺ CD8⁺ T cells with low or high MMP from patients with advanced lung cancer and sarcoma (n=17) as measured by CD107a expression comparing baseline (BL), 24 hours, 1 week, and 2 weeks post SFRT therapy (FIG. 2C), and 1 week post SFRT therapy (FIG. 2D). FIG. 2E is a pair of graphs plotting the percentages of CX3CR1⁺ (left) and CX3CR1-(right) CD8⁺ T cells with low or high MMP from patients with melanoma receiving stereotactic body radiotherapy (SBRT) at baseline (n=21). FIG. 2F is a graph plotting survival probability at baseline prior to SBRT in patients (n=21) with advanced melanoma. Kaplan-Meier estimates were used to estimate survival curves, and a univariate Cox model was used to determine the association of patients and disease variables with the risk of each outcome of interest. Statistical significance was determined by Student's Paired two-tailed t-test for FIGS. 2C and 2D, and non-paired t-test for FIG. 2E. * P<0.05, ** P<0.01, **** P<0.0001. FIGS. 2G and 2H show the change of CX3CR1⁺ CD8⁺ T cells with low MMP in response to ICI therapy. The percentage of CX3CR1⁺ CD8⁺ T cells with low MMP was measured in the peripheral blood of patients with advanced melanoma receiving anti-PD-1/PD-L1 therapy at baseline (BL) and follow up time points (N=42). The data show the trends of the frequency of CX3CR1⁺ CD8⁺ T cells with low MMP in responders (FIG. 2G) and non-responders (FIG. 2H).

FIGS. 3A-3H show that resilient CD8 T cells have high cytolytic capability. FIG. 3A is a diagram of the experimental schematics. FIG. 3B is a heatmap of differentially expressed genes from bulk RNAseq of anti-CD3/CD28 activated CD8⁺ T cells sorted according to low and high MMP (N=3). The framed area indicates genes encoding cytotoxic molecules. FIG. 3C is a graph plotting relative GZMB and NKG7 mRNA expression in 5 donors. FIG. 3D is a pair of graphs plotting expression of granzyme B protein in activated CD8⁺ T cells with low or high MMP as analyzed by flow cytometry for MFI (median fluorescent intensity; left) and percentage of positive cells (right). FIG. 3E is a pair of graphs plotting the results of a cytotoxicity assay for CD8⁺ T cells with low or high MMP against tumor cells (MCF-7 breast cancer, left; PC.3 prostate cancer, right) at a ratio of 1:20 (Tumor:Effector) in a 4-hour Calcein release assay (n=9-10). FIG. 3F is a graph plotting the results of a degranulation assay for healthy donor CX3CR1+CD8⁺ T cells with low or high MMP as measured by CD107a expression (n=6). FIG. 3G shows the results of Hallmark Gene Set Enrichment Analysis (GSEA) of RNAseq data from three healthy donors using IPA analysis software. FIG. 3H shows the results of C7 immunological pathyway analysis for effector gene signatures in low and high MMP cells. Statistical significance was determined by Student's paired t-test for FIG. 3D-3F or non-paired two-tailed t-test for FIG. 3C. * P<0.05, ** P<0.01.

FIGS. 4A-4F show that resilient CD8 T cells are not prone to be exhausted. FIG. 4A includes a pair of graphs plotting PD-1 expression by activated CD8⁺ T cells with low and high MMP, shown as percent of positive cells (left) and median fluorescent intensity (MFI, right). FIG. 4B is a graph plotting TOX expression by activated CD8⁺ T cells with low and high MMP as MFI. FIG. 4C is a graph plotting the percentage of TCF-1⁺ PD-1⁺ CD8⁺ T cells in low and high MMP cells at activated state. FIG. 4D is a pair of graphs plotting cytotoxicity in CD8⁺ T cells with low and high MMP that were activated with anti-CD3/CD28 beads in the presence of anti-PD-1 (Pembro) or PD-L1 (Atezo) for 72 hours, followed with a 4 hour calcein release cytotoxicity assay at a ratio of 1:20 (Tumor: Effector) against PC.3 tumor cells (n=7-13). FIG. 4E is a pair of box plots of bulk RNA seq transcripts for Eomes, T-bet, and CX3CR1, and FIG. 4F is a pair of graphs plotting the results of flow cytometry analysis of Eomes and T-bet in CD8⁺ T cells with low or high MMP at activated state (n=6). Statistical significance was determined by Student's Paired t-test for FIG. 4A and by non-paired two-tailed t-test for FIGS. 4B-4D and 4F. *P<0.05, ** P<0.01.

FIGS. 5A-5G show that resilient CD8 T cells have lower glycolysis and metabolic fitness via mitochondrial ATP production. FIGS. 5A and 5B include graphs plotting levels of glycolysis and glycolytic capacity (FIG. 5A) of sorted CD8⁺ T cells with low and high MMP after activation with anti-CD3/CD28 beads as measured with extracellular acidification rate (ECAR) analysis and shown with a representative of Seahorse graph (FIG. 5B, n=6). FIG. 5C is a pair of graphs plotting basal respiration (left) and spare respiratory capacity (right) of sorted CD8⁺ T cells with low and high MMP after activation as measured with Mitochondrial Oxygen Consumption Rate (OCR) analysis. FIG. 5D is a representative graph of OCR as measured by MitoStress Test (n=6). FIG. 5E is a graph plotting mitochondrial ATP of activated CD8⁺ T cells obtained from Mitochondrial Oxygen Consumption Rate (OCR) analysis (n=6). FIG. 5F is a graph plotting OCR/ECAR ratio calculated from the basal respiration values (n=6). FIG. 5G is a graph plotting GLUT1 expression (MFI) in activated CD8⁺ T cells with low and high MMP (n=4). Statistical significance was determined by Student's paired two-tailed t-test. * P<0.05.

FIGS. 6A-6G show that resilient CD8 T cells have a stable lower ROS in cytosol and mitochondria. FIG. 6A shows Hallmark GSEA analysis of ROS pathway genes from bulk RNA-seq data. FIG. 6B is a pair of graphs plotting cytosol ROS measured by flow cytometry using CELLROX™ Green and shown as MFI in both resting (left) and activated (right) CD8⁺ T cells. FIG. 6C is a graph plotting mitochondria ROS measured by flow cytometry using MITOSOX™ over one week in culture media. FIG. 6D is a graph plotting mitochondrial mass measured using MITOTRACKER™ Green with flow cytometry and shown as MFI. FIG. 6E is a graph plotting the number of mitochondria per cell counted via transmission electron microscopy (TEM) among 10-16 view fields. FIGS. 6F and 6G includes graphs plotting ROS levels in CD8⁺ T cells with low or high MMP, measured from peripheral blood mononuclear cells (PBMCs) isolated from patients with lung cancer or sarcoma receiving SFRT (FIG. 6F, n=9), and from patient patients with prostate cancer receiving SBRT (FIG. 6G, n=21). Statistical significance was determined with Student's Non-paired two-tailed t-test. ** P<0.01, *** P<0.001, *P<0.0001.

FIGS. 7A-7H show that resilient CD8 T cells express more ME1 that can reduce ROS and promote CTL function. FIG. 7A is a pair of volcano plots of bulk RNA-seq of CD8⁺ T cells with low or high MMP at resting (left) and activated (right) states (n=3; ME1 is circled). FIG. 7B is a pair of box plots showing the results of ME1 expression analysis with bulk RNA-seq as in FIG. 7A. FIG. 7C is a graph plotting relative ME1 mRNA expression measured with qRT-PCR (n=6). FIG. 7D is an image of a Western blot of ME1 expression as in FIG. 7C for two donors. FIG. 7E is a schematic for a functional study of T cells with overexpression of ME1. FIGS. 7F and 7G include graphs plotting ME1 expression measured by qRT-PCR (FIG. 7F), and an image of a representative Western blot (FIG. 7G) for CD8⁻ T cells after transfection with control or ME1 mRNA. FIG. 7H is a pair of graphs plotting ROS levels measured in CD8⁺ T cells with low (left) or high (right) MMP isolated from healthy donors (n=3) after transfection with control or ME1 mRNA. Statistical significance was determined by Student's Paired t-test. * P<0.05, ** P<0.01.

FIGS. 8A-8K show that ME1 overexpression increases CD8⁺ T cell cytotoxicity and ATP production without increasing cytosolic and mitochondrial ROS. FIG. 8A is a graph plotting the cytotoxicity of CD8⁺ T cells isolated from healthy donors (n=6) after transfection with control or ME1 mRNA, in killing tumor cells (MCF-7) at a ratio of 1:10 of tumor: effector cells. FIG. 8B is a pair of graphs plotting percent CX3CR1⁺ NKG7⁺ cells and percent CX3CR1⁺ GZMB⁺ cells among CD8⁺ T cells after transfection with control or ME1 mRNA, measured by flow cytometry (n=4). FIG. 8C shows PD-1 levels (MFI) in CD8⁺ T cells after ME1 mRNA transfection (n=6). FIG. 8D is a heatmap of differentially expressed genes from bulk RNAseq of anti-CD3/CD28 activated CD8⁺ T cells after transfection with control or ME1 mRNA (n=3). FIG. 8E is a graph plotting the cytotoxicity of CD8⁺ T cells isolated from healthy donors (n=7) after transfection with control or ME1 mRNA; and treatment with JAK1 inhibitor (upadacitinib), with DMSO as control, in killing tumor cells (MCF-7) at a ratio of 1:10 of tumor: effector cells. FIG. 8F is a graph plotting of seahorse analysis of mitochondria respiration as shown by the representative graph (left) and basal and maximal respiration (top right), and ATP-linked respiration and spare respiratory capacity (bottom right) in CD8⁺ T cells following transfection with control or ME1 mRNA (n=6). FIGS. 8G-8H show cytosolic ROS (FIG. 8G) and mitochondrial ROS (FIG. 8H) in activated CD8⁺ T cells following transfection with control or ME1 mRNA as measured by flow cytometry (MFI). FIG. 8I shows differential central carbon metabolites in ME1 overexpressing CD8⁺ T cells compared to control mRNA transfected CD8⁺ T cells as measured by LC/MS. FIG. 8J is a graph plotting of NADPH concentration per 1×10⁶ was measured at 24 hours post activation in control mRNA or ME1 mRNA transfected CD8⁺ T cells (n=5). FIG. 8K is a graph plotting of seahorse analysis of extracellular acidification rate (ECAR), quantification of glycolytic capacity and glycolysis in CD8⁺ T cells transfected with ME1 mRNA or control mRNA (n=6). Statistical significance was determined by Student's Paired t-test for FIGS. 8A-8C, 8F, 8J, and 8K or non-paired two-tailed t-test for FIGS. 8G-8I. * P<0.05, ** P<0.01, *** P<0.001.

FIGS. 9A-9E show that ME1 can increase the CTL function of peripheral blood lymphocytes of patients with advanced disease. FIG. 9A is a pair of graphs plotting ROS levels (MFI) measured in CD8⁺ T cells with low (left) or high (right) MMP among PBMCs of patients (n=5) with advanced cancer (prostate cancer), after transfection of the T cells with control or ME1 mRNA followed by T cell activation by anti-CD3 antibody for 48 hours. FIG. 9B is a pair of graphs plotting relative cytotoxicity of peripheral lymphocytes transfected with ME1 mRNA vs. control mRNA normalized to 1 among patient specimens that are identified as responders or non-responders to ME1 overexpression in context of increased cytotoxicity in prostate and melanoma patients' cells. The cytotoxicity assay was performed at a ratio of 1:10 of tumor: effector cells. FIG. 9C is a graph plotting of flow cytometry analysis of ME1 expression in patients peripheral blood lymphocytes in ME1 mRNA vs. control mRNA (n=6). FIG. 9D is an image of a Western blot showing ME1 knockdown in CD8⁺ T cells after transfection with control or ME1 siRNA. FIG. 9E is a graph plotting the results of a cytotoxicity assay of CD8⁺ T cells with low of high MMP after transfection with control or ME1 siRNA at a ratio of 1:10 of tumor: effector cells. Statistical significance was determined by Student's Paired t-test for FIGS. 9B-9C, or non-paired two-tailed t-test for FIGS. 9A and 9E. *P<0.05.

### DETAILED DESCRIPTION

As described herein, resilient T cells can explain the presence of highly cytotoxic T cells that are less exhausted and rebound in responses to ICI therapy. Phenotypic and functional characters of resilient T cells also are described herein. For example, resilient CD8⁺ T cells have low mitochondrial membrane potential, are highly cytotoxic, and express increased levels of ME1. This document provides compositions containing nucleic acids (e.g., vectors) that include nucleic acid sequences encoding ME1. This document also provides methods for increasing ME1 levels in cells (e.g., CD8⁺ T cells, such as CX3CR1⁺ CD8⁺ T cells), as well as methods for using cells having increased ME1 levels to treat mammals having cancer.

ME1 is a cytosolic protein that catalyzes the conversion of malate to pyruvate, simultaneously regenerating NADPH from NADP. ME1 has major roles in lipid and cholesterol biosynthesis, as it generates NADPH (a required cofactor for fatty acid and cholesterol biosynthesis). In addition, ME1 regulates the reversible oxidative decarboxylation of malate to pyruvate, thus linking the glycolytic and citric acid pathways. ME1 also participates indirectly in other NADPH-dependent metabolic pathways by virtue of its contribution to the cytosol NADPH pool. Further, ME1 has been demonstrated to be pro-oncogenic in an array of epithelial cancers. See, e.g., Simmen et al., J Mol Endocrinol 65 (4), R77-R90, 2020.

In some cases, this document provides methods for increasing the level of a polypeptide having ME1 function in a cell (e.g., a mammalian cell, such as a human cell). The methods can include introducing a nucleic acid encoding a polypeptide having ME1 function into a cell, and incubating the cell so that the nucleic acid encoding the polypeptide is expressed, thereby increasing the level of ME1 activity in the cell. Any suitable type of cell can be used. For example, the cell can be a PBMC, such as a T cell (e.g., a CTL). In some cases, the cell can be a CX3CR1⁺ CD8⁺ T cell having low mitochondrial membrane potential. It is to be appreciated that a nucleic acid can be introduced into a population of cells (e.g., a population of PBMCs), where the population includes CTLs, such as CX3CR1⁺ CD8⁺ T cells having low mitochondrial membrane potential. Any appropriate method for obtaining PBMCs or CTLs can be used, including those described herein. For example, PMBCs can be isolated from donor blood by centrifugation with LYMPHOPREP™ (STEMCELL Technologies), and CD8⁻ T cells can be isolated from a PBMC preparation using anti-CD8 antibodies or a commercially available kit (e.g., a magnet-based CD8 T cell isolation kit available from STEMCELL Technologies).

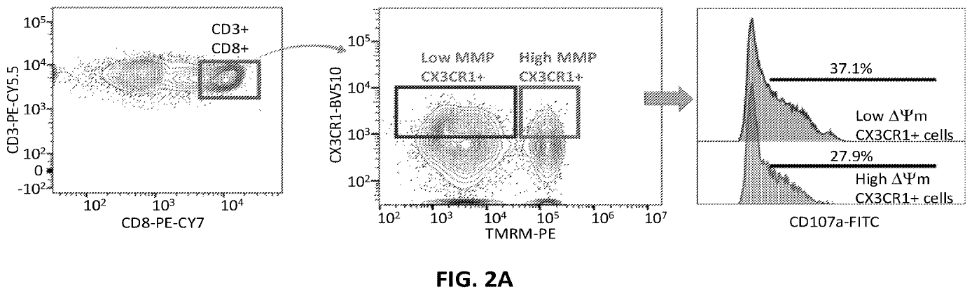

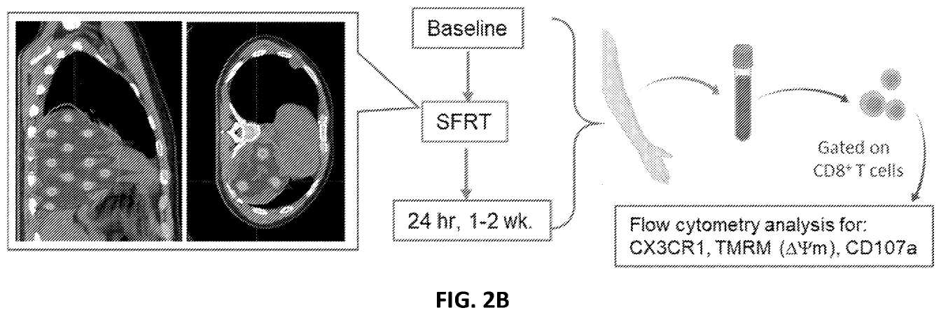

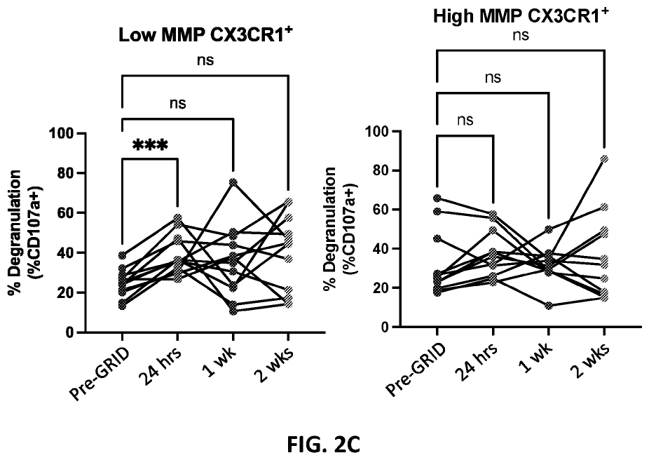

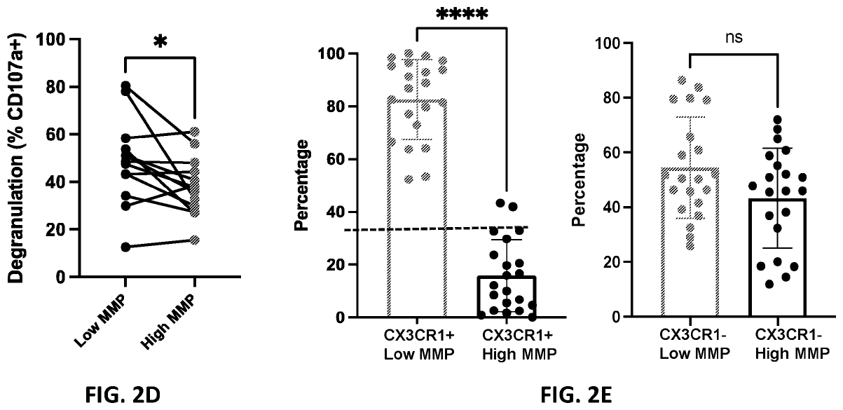

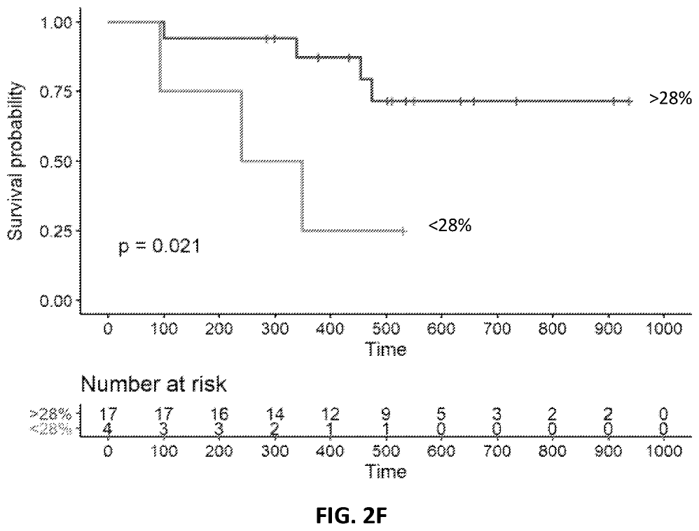

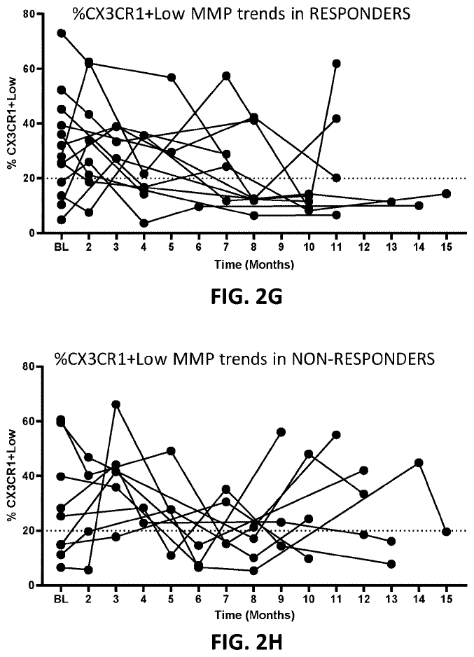

The mitochondrial membrane potential of cells can be determined using any appropriate method. As described in Example 1 and shown in FIG. 2A, for example, mitochondrial membrane potential can be assessed using tetramethyl rhodamine methyl ester (TMRM) dye, followed by flow cytometry analysis. Other cell membrane permeable fluorescent dyes also can be used to assess mitochondrial membrane potential. Such dyes include, without limitation, 3, 3′-dihexyloxacarbocyanine iodide (DiOC₆), rhodamine-123 (Rh123), tetramethyl rhodamine ethyl ester (TMRE), and JC-1. As used herein, “low” mitochondrial membrane potential indicates a lower energy capacity of the inner mitochondrial membrane and potentially lower synthesis of ATP, while “high” mitochondrial membrane potential indicates that the mitochondrial respiratory chain becomes a significant producer of reactive oxygen species (ROS) (see, e.g., Zorova et al., Analytic Biochemistry, 552, 50-59, 2018). Without being bound by a particular mechanism, ME1 upregulation in cells with low mitochondrial membrane potential may allow production of required amounts of ATP and also of lower levels of ROS that would not be harmful to cells.

Once the desired type of cells has been obtained, nucleic acid encoding ME1 can be introduced into the cells. As used herein, the term “nucleic acid” encompasses RNA and DNA, including cDNA, genomic DNA, and synthetic (e.g., chemically synthesized) DNA. The nucleic acid can be circular or linear, and can be double-stranded or single-stranded. Where single-stranded, the nucleic acid can be the sense strand or the antisense strand. In the methods provided herein, the nucleic acid introduced into a cell can be a DNA or an RNA.

The term “isolated” as used herein with reference to nucleic acid refers to a naturally-occurring nucleic acid sequence that is not immediately contiguous with both of the sequences with which it is immediately contiguous (one on the 5′ end and one on the 3′ end) in the naturally-occurring genome of the organism from which it is derived. For example, an isolated nucleic acid can be, without limitation, a recombinant DNA molecule of any length, provided one of the nucleic acid sequences normally found immediately flanking that recombinant DNA molecule in a naturally-occurring genome is removed or absent. Thus, an isolated nucleic acid includes, without limitation, a recombinant DNA that exists as a separate molecule (e.g., a cDNA or a genomic DNA fragment produced by PCR or restriction endonuclease treatment) independent of other sequences as well as recombinant DNA that is incorporated into a vector, an autonomously replicating plasmid, a virus (e.g., a retrovirus, adenovirus, or herpes virus), or into the genomic DNA of a prokaryote or eukaryote. In addition, an isolated nucleic acid can include a recombinant DNA molecule that is part of a hybrid or fusion nucleic acid sequence.

The term “isolated” as used herein with reference to nucleic acid also includes any non-naturally-occurring nucleic acid since non-naturally-occurring nucleic acid sequences are not found in nature and do not have immediately contiguous sequences in a naturally-occurring genome. For example, non-naturally-occurring nucleic acid such as an engineered nucleic acid is considered to be isolated nucleic acid. Engineered nucleic acid can be made using common molecular cloning or chemical nucleic acid synthesis techniques. Isolated non-naturally-occurring nucleic acid can be independent of other sequences, or incorporated into a vector, an autonomously replicating plasmid, a virus (e.g., a retrovirus, adenovirus, or herpes virus), or the genomic DNA of a prokaryote or eukaryote. In addition, a non-naturally-occurring nucleic acid can include a nucleic acid molecule that is part of a hybrid or fusion nucleic acid sequence.

It will be apparent to those of skill in the art that a nucleic acid existing among hundreds to millions of other nucleic acid molecules within, for example, cDNA or genomic libraries, or gel slices containing a genomic DNA restriction digest is not to be considered an isolated nucleic acid.

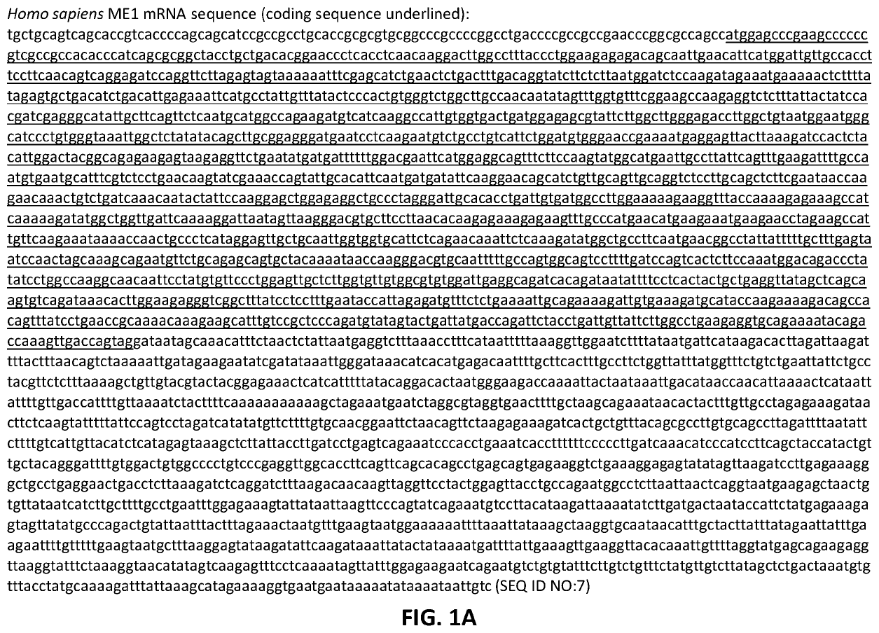

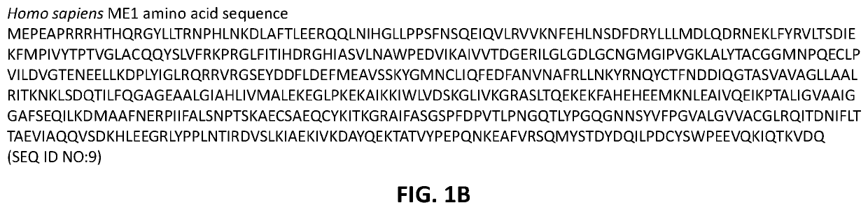

In some cases, a nucleic acid used in the methods provided herein can encode human ME1. A representative example of a human ME1 mRNA sequence is set forth in SEQ ID NO:7 (FIG. 1A). The ME1 coding sequence (SEQ ID NO:8) within SEQ ID NO: 7 is underlined in FIG. 1A. In some cases, the nucleic acid encoding ME1 can include one or more (e.g., two, three, four, five, six, seven, eight, nine, ten, or more than ten) sequence variations (e.g., additions, deletions, and/or substitutions) as compared to SEQ ID NO:7 or SEQ ID NO:8. For example, a nucleic acid introduced into a cell can have a nucleotide sequence that is less than 100% identical to SEQ ID NO:7 or SEQ ID NO: 8, but is at least 90% (e.g., at least 93%, at least 95%, at least 96%, at least 97%, at least 98%, or at least 99%) identical to the sequence set forth in SEQ ID NO:7 or SEQ ID NO: 8. A representative human ME1 amino acid sequence is set forth in SEQ ID NO:9 (FIG. 1B). In some cases, the nucleic acid encoding ME1 can encode a polypeptide having the sequence set forth in SEQ ID NO:9. In some cases, the amino acid sequence of the encoded ME1 polypeptide can include one or more amino acid sequence additions, deletions, or substitutions as compared to SEQ ID NO:9. For example, a ME1 polypeptide expressed in a cell can have an amino acid sequence that is less than 100% identical to SEQ ID NO:9, but is at least 90% (e.g., at least 93%, at least 95%, at least 96%, at least 97%, at least 98%, or at least 99%) identical to the sequence set forth in SEQ ID NO:9. In some cases, a ME1 polypeptide can have an amino acid substitution that can prevent ubiquitin-mediated degradation of the polypeptide, thereby increasing the level of the polypeptide with a cell. For example, amino acids (e.g., lysine residues) to which ubiquitin enzymes can attach can be substituted with other amino acid (e.g., alanine residues), such that the polypeptide is not tagged for ubiquitination and cannot be targeted for degradation.

The percent sequence identity between a particular amino acid or nucleic acid sequence and an amino acid or nucleic acid sequence referenced by a particular sequence identification number is determined as follows. First, an amino acid or nucleic acid sequence is compared to the sequence set forth in a particular sequence identification number using the BLAST 2 Sequences (B12seq) program from the stand-alone version of BLASTZ containing BLASTN version 2.0.14 and BLASTP version 2.0.14. This stand-alone version of BLASTZ can be obtained from Fish & Richardson's web site (e.g., www.fr.com/blast/) or the U.S. government's National Center for Biotechnology Information web site (www.ncbi.nlm.nih.gov). Instructions explaining how to use the B12seq program can be found in the readme file accompanying BLASTZ. B12seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to −1; -r is set to 2; and all other options are left at their default setting. For example, the following command can be used to generate an output file containing a comparison between two sequences: C:\B12seq-i c:\seq1.txt -j c:\seq2.txt -p blastn -o c:\output.txt -q −1 -r 2. To compare two amino acid sequences, the options of B12seq are set as follows: -i is set to a file containing the first amino acid sequence to be compared (e.g., C:\seq1.txt) ; -j is set to a file containing the second amino acid sequence to be compared (e.g., C:\seq2.txt) ; -p is set to blastp; -o is set to any desired file name (e.g., C:\output.txt); and all other options are left at their default setting. For example, the following command can be used to generate an output file containing a comparison between two amino acid sequences: C:\B12seq-i c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. A matched position refers to a position in which an identical nucleotide or amino acid residue occurs at the same position in aligned sequences. The percent sequence identity is determined by dividing the number of matches by the length of the sequence set forth in the identified sequence (e.g., SEQ ID NO:8), followed by multiplying the resulting value by 100. For example, a nucleotide sequence that has 1700 matches when aligned with the sequence set forth in SEQ ID NO:8 is 98.9 percent identical to the sequence set forth in SEQ ID NO:8 (i.e., 1700÷1719×100=98.9). It is noted that the percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. It also is noted that the length value will always be an integer.

In some cases, a nucleic acid encoding ME1 can be included in a vector that is introduced into a cell. A “vector” is a replicon, such as a plasmid, phage, or cosmid, into which another DNA segment may be inserted so as to bring about the replication of the inserted segment. An “expression vector” is a vector that includes one or more expression control sequences, and an “expression control sequence” is a DNA sequence that controls and regulates the transcription and/or translation of another DNA sequence.

In an expression vector, a nucleic acid (e.g., a nucleic acid encoding a chimeric polypeptide provided herein) can be operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 to 500 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalovirus, retroviruses, vaccinia viruses, adenoviruses, and adeno-associated viruses. Numerous vectors and expression systems are commercially available from such corporations as Novagen (Madison, WI), Clontech (Palo Alto, CA), Stratagene (La Jolla, CA), and Invitrogen/Life Technologies (Carlsbad, CA).

Any appropriate method can be used to introduce a nucleic acid encoding ME1 into a cell in vivo or in vitro. RNA (e.g., mRNA) can be introduced using, for example, nucleofection (e.g., as described in Example 1 herein). In some cases, mRNA can be delivered using a viral vector that carries a cDNA encoding ME1 for transcription of ME1 mRNA, thus generating modified T cells that overexpress ME1. Other suitable methods for introducing nucleic acids into cells can be found, for example, in Sambrook et al., Molecular Cloning: A Laboratory Manual (2^{nd} edition), Cold Spring Harbor Laboratory, New York (1989). For example, calcium phosphate precipitation, electroporation, heat shock, lipofection, microinjection, and viral-mediated nucleic acid transfer can be used introduce nucleic acid into cells. In addition, naked DNA can be delivered directly to cells in vivo as described elsewhere (U.S. Pat. Nos. 5,580,859 and 5,589,466). The host cells can express an encoded polypeptide, but it is noted that cells containing an isolated nucleic acid molecule provided herein are not required to express a polypeptide. An isolated nucleic acid molecule transformed into a host cell can be integrated into the genome of the cell or maintained in an episomal state. Thus, host cells can be stably or transiently transfected with a construct containing an isolated nucleic acid molecule provided herein.

Any appropriate method can be used to identify cells containing an introduced (exogenous) nucleic acid molecule or vector provided herein, and/or to identify cells having an increased level of ME1 as a result of the introduced nucleic acid. Such methods include, without limitation, PCR and nucleic acid hybridization techniques such as Northern and Southern analyses. In some cases, immunohistochemistry and/or biochemical techniques can be used to determine if a cell contains a particular isolated nucleic acid molecule by detecting the expression and/or the level of a polypeptide encoded by that nucleic acid molecule. The term “exogenous” as used herein with reference to a nucleic acid introduced into a cell refers to a nucleic acid molecule that did not originate within the cell, although the exogenous nucleic acid can include a nucleotide sequence that is found within the cell. For example, an exogenous nucleic acid can include a human ME1 coding sequence, and can be introduced into a human cell.

As used herein, an “increased” or “elevated” level of ME1 refers to any level of ME1 mRNA or ME1 polypeptide that is higher than a reference level of the ME1 mRNA or polypeptide. The term “reference level” as used herein with respect to an ME1 mRNA or ME1 polypeptide refers to the level of the ME1 mRNA or polypeptide typically observed in control samples. Control samples can include, without limitation, cells that do not contain an introduced ME1 nucleic acid. For example, the level of ME1 mRNA or ME1 polypeptide in a population of cells (e.g., PBMCs) containing an introduced nucleic acid encoding ME1 can be considered to be “increased” if the level is at least 5% (e.g., at least 10%, at least 15%, at least 20%, at least 25%, at least 30%, at least 40%, at least 50%, at least 75%, or more than 100%) greater than a reference level of the ME1 mRNA or ME1 polypeptide in a control sample (e.g., a corresponding population of cells, such as PBMCs, that do not contain the introduced nucleic acid encoding ME1). It will be appreciated that levels of an ME1 mRNA or ME1 polypeptide from comparable samples are used when determining whether or not a particular level is an increased level of the mRNA or polypeptide.

Any appropriate method can be used to detect the presence or absence of an increased level of an ME1 mRNA or ME1 polypeptide in a sample (e.g., a sample containing a population of cells). In some cases, the presence, absence, or level of an ME1 mRNA within a sample can be determined by detecting mRNA encoding an ME1 polypeptide in the sample. For example, polymerase chain reaction (PCR)-based techniques such as quantitative RT-PCR techniques, gene expression panel (e.g., next generation sequencing (NGS) such as RNA-seq), in situ hybridization, and/or microarray gene expression profiling can be used to determine the presence, absence, or level of ME1 mRNA in the sample. In some cases, the presence or absence of an increased level of an ME1 polypeptide within a sample can be determined by detecting the presence, absence, or level of the ME1 polypeptide in the sample. For example, immunoassays (e.g., immunohistochemistry (IHC) techniques and western blotting techniques), mass spectrometry techniques (e.g., proteomics-based mass spectrometry assays or targeted quantification-based mass spectrometry assays such as liquid chromatography-tandem mass spectrometry (LC-MS/MS)), enzyme-linked immunosorbent assays (ELISAs), radio-immunoassays, and/or immunofluorescent cytochemistry (IFC) can be used to determine the presence, absence, or level of an ME1 polypeptide in a sample. When an immunoassay is used to determine the presence, absence, or level of an ME1 polypeptide in a sample, the immunoassay can include using any appropriate anti-ME1 antibody. Examples of anti-ME1 antibodies that can be used in an immunoassay (e.g., IFC or ELISA) to determine the presence, absence, or level of a ME1 polypeptide in a sample include, for example, antibodies that are commercially available (e.g., anti-human ME1 antibodies ab97445 and ab223761 from Abcam, Cambridge, UK; and antibodies PA5-21550, MA5-23524, PA5-40660, PA5-82251, MA5-49254, MA5-27763, and MA5-27762 from ThermoFisher Scientific, Waltham, MA). In some cases, a level of ME1 can be assessed based on ME1 activity.

This document also provides compositions containing cells having elevated expression of ME1, for administration to a subject (e.g., a mammal having cancer). For example, provided herein are compositions containing cells into which an exogenous nucleic acid encoding ME1 has been introduced, as described herein. In some cases, for example, the cells can be PBMCs. In some cases, the cells can be CTLs, such as CX3CR1⁺ CTLs. In some cases, the CX3CR1⁺ CTLs containing an introduced nucleic acid encoding ME1 can have low mitochondrial membrane potential. The nucleic acid can be RNA or DNA. In some cases, for example, the nucleic acid can include a sequence having at least 90% identity to the sequence set forth in SEQ ID NO:8.

In addition to cells having elevated expression of ME1, the compositions provided herein can include one or more agents (e.g., cytokines) that can promote T cell activation (e.g., IL-2), T cell proliferation (e.g., IL-15), and/or T cell survival (e.g., IL-7), which may facilitate or enhance ME1 expression in the T cells. In some cases, for example, a composition can contain cells as provided herein in combination with a pharmaceutically acceptable carrier. Pharmaceutically acceptable carriers include, for example, pharmaceutically acceptable solvents, suspending agents, or any other pharmacologically inert vehicles for delivering cells to a subject.

This document also provides methods for treating a mammal (e.g., a human having cancer). The methods can include, for example, administering to a mammal (e.g., a human having cancer) a composition that contains cells into which an exogenous nucleic acid encoding ME1 was introduced, such that the cells have an elevated level of ME1. Any appropriate mammal can be treated as described herein. For example, humans or other primates such as monkeys can be administered a composition containing cells having increased ME1 expression. In some cases, dogs, cats, horses, cows, pigs, sheep, rabbits, mice, or rats can be administered a composition containing cells having increased ME1 expression, as described herein.

In some cases, the cells administered to a mammal can have been obtained from a mammal (e.g., a mammal having cancer), and can have been subjected to introduction of a nucleic acid encoding ME1 before being administered back to the mammal.

A mammal treated according to the methods provided herein can be identified as having any appropriate type of cancer. For example, a mammal treated as described herein can have a cancer such as lung cancer, breast cancer, prostate cancer, colorectal cancer, gastric cancer, pancreatic cancer, melanoma, sarcoma, or ovarian cancer.

A composition containing cells with increased expression of ME1 can be administered to mammal by any appropriate route. Administration can be, for example, parenteral (e.g., by intrathecal, intraventricular, intramuscular, intrapleural, or intraperitoneal injection, or by intravenous (i.v.) drip). Administration can be rapid (e.g., by injection) or can occur over a period of time (e.g., by slow infusion). Compositions for parenteral administration can sterile aqueous solutions, which also can contain buffers, diluents, and/or other suitable additives (e.g., penetration enhancers, carrier compounds and other pharmaceutically acceptable carriers).

Methods for treating a mammal (e.g., a human) having cancer can include administering, to the mammal, an effective amount of a composition cells with increased ME1 expression. In some cases, an effective amount of a composition (e.g., a pharmaceutical composition provided herein) containing cells described herein can be an amount that reduces one or more symptoms associated with a cancer within a mammal, reduces the number of tumor cells within a mammal, reduces the size of a tumor within the mammal, or prolongs progression free survival, recurrence free survival, and/or overall survival of the mammal, without producing significant toxicity to the mammal. In some cases, an effective amount of a composition containing cells described herein (e.g., a pharmaceutical composition provided herein) can be an amount that reduces one or more symptoms associated with a cancer in a mammal as compared to a control mammal having a comparable cancer and not treated with the composition. For example, an effective amount of a composition described herein can be an amount that contains from about 10⁸ cells to about 10¹⁰ cells (e.g., about 10⁸ to about 10⁹ cells, or about 10⁹ to about 10¹⁰ cells). The effective amount can remain constant or can be adjusted as a sliding scale or variable dose depending on the mammal's response to treatment. Various factors can influence the actual effective amount used for a particular application. For example, the severity of the cancer when treating a mammal having such a disease, the route of administration, the age and general health condition of the mammal, excipient usage, the possibility of co-usage with other therapeutic treatments such as use of other anti-cancer agents (e.g., chemotherapy drugs), and the judgment of the treating physician may require an increase or decrease in the actual effective amount of a composition provided herein that is administered. After treatment, the mammal can be monitored for both responsiveness to the treatment and toxicity symptoms. If a particular mammal fails to respond to a particular amount, then the number of cells administered can be increased by, for example, two-fold. After receiving the higher number of cells, the mammal can be further monitored for both responsiveness to the treatment and toxicity symptoms, and further adjustments made accordingly.

In some cases, an effective frequency of administration of a composition containing cells with increased ME1 expression as described herein can be a frequency that reduces one or more symptoms associated with a cancer in the mammal, reduces the number of tumor cells within the mammal, reduces the size of a tumor within the mammal, or prolongs progression free survival, recurrence free survival, and/or overall survival of the mammal, without producing significant toxicity to the mammal. In some cases, an effective frequency of administration of a composition containing cells described herein (e.g., a pharmaceutical composition provided herein) can be a frequency that reduces one or more symptoms associated with a cancer in a mammal as compared to a control mammal having a comparable cancer and not treated with the composition. For example, an effective frequency of administration of a pharmaceutical composition described herein can be from about twice a week to about once a month (e.g., once a week, once every 14 days, once every 21 days, or once every 28 days). The frequency of administration of a pharmaceutical composition described herein such as a pharmaceutical composition containing cells described herein can remain constant or can be variable during the duration of treatment. Various factors can influence the actual effective frequency used for a particular application. For example, the effective amount, the severity of the cancer when treating a mammal having such a cancer, the route of administration, the age and general health condition of the mammal, excipient usage, the possibility of co-usage with other therapeutic or prophylactic treatments such as use of other anti-cancer agents (e.g., chemotherapy drugs or checkpoint inhibitors), and the judgment of the treating physician may require an increase or decrease in the actual effective frequency of administration of a composition provided herein (e.g., a pharmaceutical composition containing cells having increased ME1 expression as described herein).

In some cases, an effective duration of administration of a composition (e.g., a pharmaceutical composition provided herein) containing cells described herein can be a duration that reduces one or more symptoms associated with a cancer in a mammal, reduces the number of tumor cells within a mammal, reduces the size of a tumor within the mammal, or prolongs progression free survival, recurrence free survival, and/or overall survival of the mammal, without producing significant toxicity to the mammal. In some cases, an effective duration of administration of a composition containing cells described herein (e.g., a pharmaceutical composition provided herein) can be a duration that reduces one or more symptoms associated with a cancer in a mammal having such cancer as compared to a control mammal having a comparable cancer and not treated with the composition. For example, an effective duration of administration of a pharmaceutical composition provided herein, such as a pharmaceutical composition containing cells with increased ME1 expression can vary from a single time point of administration to administration over the course of several weeks to several months (e.g., 2 to 4 weeks, 4 to 8 weeks, 8 to 12 weeks, 12 to 16 weeks, or more than 16 weeks). Multiple factors can influence the actual effective duration used for a particular application. For example, the severity of the cancer, the effective frequency, the effective amount, the route of administration, the age and general health condition of the mammal, excipient usage, the possibility of co-usage with other therapeutic or prophylactic treatments such as use of other anti-cancer agents (e.g., chemotherapeutic agents), and the judgment of the treating physician may require an increase or decrease in the actual effective duration of administration of a composition provided herein (e.g., a pharmaceutical composition containing cells described herein).

In some cases, when treating a mammal (e.g., a human) having cancer as described herein, the treatment can be effective to treat the cancer. For example, cancer progression within a mammal can be slowed using the methods and materials described herein. In some cases, the methods and materials described herein can be used to slow cancer progression within a mammal having cancer by, for example 10, 20, 30, 40, 50, 60, 70, 80, 90, 95, or more percent. In some cases, the cancer does not progress. In some cases, tumor growth can be slowed using the methods and materials described herein. In some cases, the methods and materials described herein can be used to slow the growth of a tumor in a mammal having cancer by, for example, 10, 20, 30, 40, 50, 60, 70, 80, 90, 95, or more percent. In some cases, tumor growth in the mammal does not occur. In some cases, the methods and materials described herein can be used to reduce the number of tumor cells in a mammal having cancer. For example, the methods and materials described herein can be used to reduce the number of tumor cells in a mammal by 10, 20, 30, 40, 50, 60, 70, 80, 90, 95, or more percent.

In some cases, when treating a mammal (e.g., a human) having cancer as described herein, the treatment can be effective to prolong periods of remission. For example, the methods and materials described herein can be used to prolong periods of disease remission in a mammal having cancer by, for example, 10, 20, 30, 40, 50, 60, 70, 80, 90, 95, or more percent. For example, the methods and materials described herein can be used to prolong periods of cancer remission in a mammal by, for example, at least 6 months (e.g., about 6 months, about 8 months, about 10 months, about 1 year, about 1.5 years, about 2 years, about 2.5 years, about 3 years, or more than about 3 years).

In some cases, when treating a mammal (e.g., a human) having cancer as described herein, the treatment can be effective to improve survival of the mammal. For example, the methods and materials described herein can be used to improve progression-free survival, recurrence-free survival, and/or overall survival. For example, the methods and materials described herein can be used to increase the survival (e.g., progression-free survival, recurrence-free survival, and/or overall survival) of a mammal having cancer by, for example, 10, 20, 30, 40, 50, 60, 70, 80, 90, 95, or more percent. For example, the methods and materials described herein can be used to improve the survival (e.g., progression-free survival, recurrence-free survival, and/or overall survival) of a mammal having cancer by, for example, at least 6 months (e.g., about 6 months, about 8 months, about 10 months, about 1 year, about 1.5 years, about 2 years, about 2.5 years, or about 3 years).

In some cases, the methods provided herein can include monitoring a mammal after treatment with cells having increased expression of ME1, to assess the effectiveness of the treatment. In some cases, for example, a course of treatment and/or the severity of one or more symptoms related to the cancer being treated can be monitored. Any appropriate method can be used to determine whether a mammal having cancer is responding to treatment. For example, clinical scanning techniques (e.g., computed tomography (CT), positron emission tomography (PET)/CT, bone scan, and magnetic resonance imaging (MRI)) can be used to assess the presence, absence, or physical characteristics (e.g., size) of a cancer within a mammal (e.g., a human) treated by the methods provided herein.

### Exemplary Embodiments

Embodiment 1 is a method for increasing a level of malic enzyme 1 (ME1) in a cell, said method comprising: introducing into said cell a nucleic acid encoding ME1, and incubating said cell such that said nucleic acid is expressed, thereby increasing the level of ME1 in said cell.

Embodiment 2 is the method of embodiment 1, wherein said cell is a T cell.

Embodiment 3 is the method of embodiment 2, wherein said T cell is a cytotoxic T lymphocyte (CTL).

Embodiment 4 is the method of embodiment 3, wherein said CTL is a CX3CR1+ CTL.

Embodiment 5 is the method of embodiment 2, wherein said T cell is a chimeric antigen receptor- (CAR-) T cell or a T cell receptor- (TCR-) T cell.

Embodiment 6 is the method of any one of embodiments 1 to 5, wherein said cell is a human cell.

Embodiment 7 is the method of any one of embodiments 1 to 6, wherein said nucleic acid is a mRNA.

Embodiment 8 is the method of any one of embodiments 1 to 7, wherein the nucleic acid encoding ME1 comprises the nucleotide sequence set forth in SEQ ID NO:8, or having a nucleotide sequence with at least 90% identity to the nucleotide sequence set forth in SEQ ID NO:8.

Embodiment 9 is a method for treating a mammal, said method comprising administering to said mammal a composition comprising cells that comprise an exogenous nucleic acid encoding ME1, such that said cells have an elevated level of ME1.

Embodiment 10 is the method of embodiment 9, wherein said mammal is a human.

Embodiment 11 is the method of embodiment 10, wherein said human has cancer. Embodiment 12 is the method of embodiment 11, wherein said cancer is lung cancer, breast cancer, prostate cancer, colorectal cancer, gastric cancer, pancreatic cancer, melanoma, sarcoma, or ovarian cancer.

Embodiment 13 is the method of any one of embodiments 9 to 12, said cells are peripheral blood mononuclear cells (PBMCs).

Embodiment 14 is the method of any one of embodiments 9 to 12, wherein are said cells are T cells.

Embodiment 15 is the method of embodiment 14, wherein said T cells are CTLs. Embodiment 16 is the method of embodiment 15, wherein said CTLs are CX3CR1+ CTLs.

Embodiment 17 is the method of any one of embodiments 9 to 16, wherein said cells were obtained from said mammal and transfected with said nucleic acid.

Embodiment 18 is the method of embodiment 14, wherein said T cells are CAR-T cells or TCR-T cells.

Embodiment 19 is the method of any one of embodiments 9 to 18, wherein said nucleic acid is a mRNA.

Embodiment 20 is the method of any one of embodiments 9 to 19, wherein the nucleic acid encoding ME1 comprises the nucleotide sequence set forth in SEQ ID NO:8, or having a nucleotide sequence with at least 90% identity to the nucleotide sequence set forth in SEQ ID NO:8.

Embodiment 21 is a composition comprising PBMCs that comprise an exogenous nucleic acid encoding ME1.

Embodiment 22 is the composition of embodiment 21, wherein said nucleic acid is a mRNA.

Embodiment 23 is the composition of embodiment 21 or embodiment 22, wherein said nucleic acid comprises the nucleotide sequence set forth in SEQ ID NO:8, or having a nucleotide sequence with at least 90% identity to the nucleotide sequence set forth in SEQ ID NO:8.

Embodiment 24 is the method of any one of embodiments 21 to 23, wherein said PBMCs comprise T cells.

Embodiment 25 is the method of embodiment 24, wherein said T cells are CTLs.

Embodiment 26 is the method of embodiment 25, wherein said CTLs are CX3CR1+ CTLs.

Embodiment 27 is a method for increasing a level of a polypeptide having malic enzyme 1 (ME1) activity in a cell, wherein said method comprises (a) introducing into said cell a nucleic acid encoding said polypeptide, and (b) incubating said cell such that said nucleic acid is expressed, thereby increasing the level of said polypeptide in said cell.

Embodiment 28 is the method of embodiment 27, wherein said polypeptide is a full-length ME1 polypeptide.

Embodiment 29 is the method of embodiment 27 or embodiment 28, wherein said polypeptide is a full-length human ME1 polypeptide.

Embodiment 30 is the method of any one of embodiments 27 to 29, wherein said polypeptide is a full-length human ME1 polypeptide comprising SEQ ID NO:8.

Embodiment 31 is the method of any one of embodiments 27 to 30, wherein said cell is a T cell.

Embodiment 32 is the method of embodiment 31, wherein said T cell is a CTL. Embodiment 33 is the method of embodiment 32, wherein said CTL is a CX3CR1+ CTL.

Embodiment 34 is the method of any one of embodiments 27 to 33, wherein said nucleic acid is a mRNA.

Embodiment 35 is a method for treating a mammal, wherein said method comprises administering to said mammal a composition comprising cells comprising an exogenous nucleic acid that encodes a polypeptide having ME1 activity, wherein said cells have an elevated level of said polypeptide.

Embodiment 36 is the method of embodiment 35, wherein said polypeptide is a full-length ME1 polypeptide.

Embodiment 37 is the method of embodiment 35 or embodiment 36, wherein said polypeptide is a full-length human ME1 polypeptide.

Embodiment 38 is the method of any one of embodiments 35 to 37, wherein said polypeptide is a full-length human ME1 polypeptide comprising SEQ ID NO:8.

Embodiment 39 is the method of any one of embodiments 35 to 38, wherein said mammal is a human.

Embodiment 40 is the method of embodiment 39, wherein said human has cancer. Embodiment 41 is the method of embodiment 40, wherein said cancer is lung cancer, breast cancer, prostate cancer, colorectal cancer, gastric cancer, pancreatic cancer, melanoma, sarcoma, or ovarian cancer.

Embodiment 42 is the method of any one of embodiments 35 to 41, said cells are PBMCs.

Embodiment 43 is the method of any one of embodiments 35 to 41, wherein are said cells are T cells.

Embodiment 44 is the method of embodiment 43, wherein said T cells are CTLs. Embodiment 45 is the method of embodiment 44, wherein said CTLs are CX3CR1+ CTLs.

Embodiment 46 is the method of embodiment 43, wherein said T cells are CAR-T cells or TCR-T cells.

Embodiment 47 is the method of any one of embodiments 35 to 46, wherein said cells were obtained from said mammal and transfected with said nucleic acid.

Embodiment 48 is the method of embodiment 47, wherein said nucleic acid is a mRNA.

The invention will be further described in the following examples, which do not limit the scope of the invention described in the claims.

### EXAMPLES

### Example 1—Highly Cytotoxic Resilient CD8⁺ T Cells Balance Extra ROS Via ME1 to Avoid Exhaustion

### Methods

CD8 T cells isolation: PMBCs as a source of peripheral lymphocytes were isolated from healthy donors or patients via centrifugation with LYMPHOPREP™ (STEMCELL Technologies) and SepMate conical tubes (STEMCELL Technologies; Vancouver, British Columbia). CD8⁺ T cells or CD8⁺ T cell subsets were then isolated using a magnet-based CD8 T cell isolation kit (STEMCELL Technologies) and used immediately for experiments. Some experiments used PBMCs (patient samples) that were stored in liquid nitrogen. For those experiments, PBMCs were thawed and incubated in CTL medium (RPMI 1640 complete medium; rhIL-2, 10 U/mL; rh IL-15, 5 ng/mL; rhIL-7, 5 ng/ml) at 37° C. overnight for recovery before transfection.

Patient information: Peripheral blood was collected after written consent was obtained from each participant. Clinical course, treatment information, and outcomes in patients treated with anti-PD-1/L1 therapy and radiation therapy were retrospectively collected. Response to treatment was evaluated according to standard clinical practice guidelines using RECIST (Yan et al., supra). The response listed for anti-PD-1/PD-L1 therapy (R-complete response, NR=progressive disease, SD=stable disease) was apparent (using RECIST) at the 12-week post-initiation of therapy time-point for all patients. For prostate cancer (receiving SFRT and SBRT), the response was evaluated based on PSA levels and distance reoccurrence. Peripheral blood from healthy people was acquired from anonymous donors.

TMRM Staining and Cell Sorting: CD8⁺ T cells were washed once with 1×PBS, adjusted to a concentration of 1×10⁶ cells/mL, and stained with 0.02 μM final concentration of tetramethylrhodamine methyl ester (TMRM) or 2 nM final concentration of carbonyl cyanide 3-chlorophenylhydrazone (CCCP) as control. The TMRM stained cells were incubated at 37° C. for 30 minutes with intermittent shaking, while the CCCP stained cells were incubated at 37° C. for 5 minutes. The cells were then washed twice with 1×PBS and resuspended at 10 to 15×10⁶ cells/mL of cell culture medium for sorting. The cells were sorted with a BD FACSMELODY™ Cell Sorter using the 100-micron sort nozzle and on the PE channel (561 nm excitation).

siRNA Transfection: Sorted CD8⁺ T cells with low and high MMP were centrifuged at 200 g for 10 minutes prior to nucleofection (4D NUCLEOFECTOR® system, Lonza). Three to five million cells were combined with 200 pMol siRNA (siControl or siME1) in 20 μL P3 nucleofection media (Lonza) per well of the 16-well NUCLEOCUVETTE® strips (X unit). Program FI-115 was used. Following nucleofection, cells were rested in warm RPMI (no FBS, no cytokines) for 4 hours before adding 10% FBS and 10 IU/mL of IL-2, 5 ng/ml of IL-7, and 5 ng/ml of IL-15 into the culture for an overnight recovery followed with use in experiments.

mRNA Transfection: T cells were transfected with 220 μg/ml control mRNA or ME1 mRNA (SEQ ID NO:8, produced at TriLink Biotechnologies, San Diego, CA) using the P3 nucleofection kit (Lonza V4XP-3024) and program FI-115 on the 4D NUCLEOFECTOR® (Lonza). Following nucleofection, cells were rested in warm RPMI (no FBS, no cytokines) for 4 hours before adding 2× CTL medium (10% FBS and IL-2, IL-7, IL-15) for an overnight recovery, followed by T cell activation and functional analysis.

Cytotoxicity Assay: Target tumor cells were first washed twice with HBSS, and then labeled with Calcein-AM (5 μM) for 30 minutes and incubated at 37° C. in the dark for 30 minutes, with occasional shaking. The cells were then washed with HBSS twice and re-suspended at 1×10⁵/mL in CTL medium with no FBS. Pre-activated CD8⁺ T cells and target cells were mixed at 1:20 or 1:10 (target to effector ratio) in CTL media without FBS and seeded into 96-U bottom well plate at 200 μL per well. Saponin (0.1%) or Triton X-100 (2%) was added to wells that contained tumor cells only to provide a value for “maximum calcein release” in each assay; tumor cell-only wells were included to measure spontaneous release of calcein. All experimental and control conditions were performed in triplicate wells. The plate was briefly centrifuged at 1000 rpm for 30 seconds, followed by incubation at 37° C. for 4 hours. After the 4-hour incubation, the plate was centrifuged at 2000 rpm for 5 minutes. 100 μL of the 200 μL supernatant was then removed from each well and added to a new, opaque (black) 96 well flat bottom plate (Thermo Scientific). Calcein fluorescence was read using an automated fluorescence measurement system (BioTeK Synergy HTX multi-mode reader) with an excitation of 485/20 and an emission filter of 530/25 scanning for 1 second per well. Percent cytotoxicity was then calculated using the formula:

$$\% Cytotoxicity = 100 \times \frac{Experimental release well - Spontaneous release well}{Maximum release well - Spontaneous release well}$$

Degranulation assay: T cells were adjusted at a concentration of 1×10⁶ cells/100 μL CTL medium including Golgi-Stop (Biolegend, 420701) and Golgi-Plug (Biolegend, 420601) and incubated with 5 μL of CD107a antibody (Biolegend, H4A3) and 5 μL of anti-CD3/CD28 beads. The cells were then briefly centrifuged at 300 g for 1 minute and incubated at 37° C. for 5 hours. After the incubation, the cells were stained with TMRM followed by surface antibody staining before flow cytometry analysis.

ROS detection: Cells were stained with 250 nM of CELLROX™ (ThermoFisher Scientific, C10492) or 1 AM of MITOSOX™ (ThermoFisher Scientific, M36008) in complete media and incubated at 37° C. for 45 minutes. From there, the cells were resuspended in FACS buffer (1×PBS, 2 mM EDTA, and 3% FBS) at a concentration of 1×10⁶ cells/100 μL followed by staining with antibodies for surface molecules for 20 minutes at room temperature in the dark. Cells were then washed once in FACS buffer, resuspended in 200 μL of FACS buffer, and analyzed on Bio-Rad ZE5 Cell Analyzer. Analysis was performed using FlowJo V10.

Flow Cytometry Analysis: Cells were adjusted to 0.5-1×10⁶ cells/mL with 1× PBS and stained with live/dead dye and incubated at 4° C. for 30 minutes. The cells were then washed once with 1×PBS followed by staining for cell surface molecules for 30 minutes at 4° C. For intracellular molecule staining, cells were incubated with FoxP3 Fixation Buffer overnight at 4° C. After fixation, cells were washed once 1× permeabilization buffer, and then stained with antibodies for intracellular molecules: ME1, TCF-1/7, TOX, Eomes, T-bet, Ki67, HIF1-α, and Glut1 for 1 hour at 4° C. in 100 μL of 1× permeabilization buffer. Following intracellular staining, cells were washed once with 1× permeabilization buffer and then resuspended in 200 μL FACS buffer for flow cytometry analysis, which was completed on a Cytoflex LX (Beckman Coulter) (DAQ Version V2.233, MCB Version: V3.01) running CytExpert software. Flow cytometric analysis was performed using FlowJo V10.

Quantitative PCR: RNA was isolated using Qiagen RNEASY® Plus Mini Kit (Qiagen). RT-reaction was completed using SUPERSCRIPT™ III Reverse Transcriptase (Invitrogen). qRT-PCR analysis was completed using a Quant-Studio 3 Real-Time PCR System (Applied Biosystems) and TaqMan Fast Advanced Master Mix (Applied Biosystems). TaqMan assays included: Hs01120688-gl, Human ME1-FAM and Hs99999901-Sl, Human-18s-FAM, both from Applied Biosystems. All samples were run in doublet. Relative expression was calculated using the 2^{-ΔCt} method. Primers used were:

Granzyme B
(SEQ ID NO: 1)
Forward 5′-TACCATTGAGTTGTGCGTGGG-3′
(SEQ ID NO: 2)
Reverse 5′-GCCATTGTTTCGTCCATAGGAGA-3′
ME1
(SEQ ID NO: 3)
Forward 5′-GGGAGACCTTGGCTGTAATGG-3′
(SEQ ID NO: 4)
Reverse 5′- TTCGGTTCCCACATCCAGAAT-3′
UBE
(SEQ ID NO: 5)
Forward 5′-GTACTCTTGTCCATCTGTTCTCTG-3′
(SEQ ID NO: 6)
Reverse 5′-CCATTCCCGAGCTATTCTGTT-3′

RNA input was normalized to 20 ng/μL with 10 μL input for the RT reaction. After the RT reaction, cDNA was diluted 1:5 before amplification on the QUANTSTUDIO™ 3 in the following volume per well: 5 μL cDNA template, 10 μL SYBR green (Applied Biosystems), 3 μL H₂O, 1 μL10 mM F/R primer.

Western Blots: Cell pellets were lysed in NP-40 buffer and concentrations were measured via protein assay using BioRad reagent (#500-0006). Once diluted to equal concentrations, samples were combined with Laemmli sample buffer (BioRad) and run using a MINI-PROTEAN® Electrophoresis and Transfer system (BioRad). Membranes were incubated with primary antibody overnight at 4° C. (ME1, Beta-Actin, or GAPDH). Secondary antibodies were added the next morning for 2 hours (HRP-conjugated anti-mouse). Signal was visualized using SUPERSIGNAL™ West Pico PLUS Chemiluminescence Substrate Kit (Thermo Fisher) on a Syngene G: Box Chemi XX6 system running GeneSys software (V1.6.1.0).

Transmission electron microscopy (TEM) analysis of mitochondria: Cells were fixed in Trump fixative for 1 hour at room temperature or at 4° C. overnight followed by fixation for 1 hour in 1% osmium tetroxide. The samples were dehydrated, embedded in Spurrs resin, sectioned at 90 nm, and observed using a Joel 1400 electron microscope (Joel USA Inc.). For quantification, images of individual T cell in a single field of view downloaded into JPEG images and the number of mitochondria structures within the T cells were counted manually by two different readers.

Metabolic Assays: Seahorse Xfe96 Bioanalyser (Agilent) was used to determine OCR and ECAR. Sorted cells were washed in XF Base media (Seahorse XF RPMI medium with 2 mM glutamine, 10 mM glucose, 1 mM sodium pyruvate, and 5 mM HEPES (4-(2-hydroxyethyl)-1-piperazineethanesulfonic acid), pH 7.4 at 37° C.) for OCR or XF media with 2 mM glutamine for ECAR before being plated onto Seahorse cell culture plates coated with CELL-TAK™ (Corning #354240) at 1×10⁵ cells per well. The cells were allowed to adhere to the culture plates. The OCR was measured using Seahorse Mito Stress assay (Agilent), with addition of oligomycin (2 μM), carbonyl cyanide 4-(trifluoromethoxy)phenylhydrazone (FCCP; 1.2 μM) and Rotenone and Antimycin (1.0 μM)). The ECAR was measured with addition of 10 mM glucose, 2 μM oligomycin, and 50 nM 2-deoxy-D-glucose (2-DG). Assay parameters were as follows: 3 minute mix, no wait, 3 minute measurement, repeated 3 to 4 times at basal and after each addition. SRC was calculated as OCR at maximum rate (OCR_{Max})−OCR in basal state (OCR_{Bas}). Mitochondrial ATP production was calculated by subtracting the minimum respiration rate after oligomycin injection from the basal respiration rate before oligomycin injection.

Central carbon metabolites on LCMS method (dMRM): CD8⁺ T cells overexpressing ME1 or control mRNA were washed twice with PBS, pelleted in Eppendorf tubes, and quickly frozen at −80° C. Central carbon metabolites (219 compounds) were monitored and measured on an Agilent 6460 triple quadrupole mass spectrometer coupled with a 1290 Infinity II quaternary pump. Acquisition was captured in negative electrospray ionization and dynamic multiple reaction monitoring (dMRM) post ion-pairing reverse phase chromatographic separation. Analytes were searched and confirmed against a curated dMRM database with retention time. Relative abundances between samples set are derived via multivariate analysis on Agilent Mass Professional Profile software (MPP).

NADPH concentration measurement: CD8⁺ T cells were transfected with either control or ME1 mRNA and rested overnight in CTL media followed by activation with anti-CD3/CD28 antibodies (STEMCELL Technologies) for 24 or 48 hours. After culture, the cells were quickly washed with cold PBS and counted. NADPH levels were measured with NADPH assay kits (Abnova, Walnut, CA).

Mitochondria Mass Staining: Cells at a concentration of 1×10⁶ cells/mL were washed once with 1×PBS and incubated with 100 nM of MITOTRACKER™ Green FM dye for 30 minutes at 37° C. The cells were then analyzed by flow cytometry.

Bulk RNA Sequencing: RNA was isolated using Qiagen RNEASY® Plus Mini Kit (Qiagen). The bulk RNA paired-end sequencing reads were processed through the Mayo bioinformatics pipelines MAP-Rseq (v3.0) as reported elsewhere (Yan et al., supra). Reads were aligned to human reference genomes (hg38). The RNA aligned reads were quantified for gene expression using the Subread package. Differences across groups were assessed using bioinformatics package edgeR 2.6.2 to identify differentially expressed genes. Such genes were reported with magnitude of change (log 2 scale) and their level of significance (False Discovery Rate, FDR <5%). For pathway analysis, T cells with low or high MMP were randomized and put through GSEA as described in the user guide.

Statistical Analyses: Data were analyzed in GraphPad Prism (version 9) using the unpaired or paired, two-tailed t-test without correction for multiple comparisons, as indicated in figure legends. Each data symbol in the drawings (e.g., circle, dot, or square) represents an average of triplicates for each healthy donor. Lines connect matched samples across all individuals in the graphs. Bar height represents mean, and error bars are SE of the mean, unless otherwise stated.

TABLE 1A
Antibody List

| | | | | | | Final |
|---|---|---|---|---|---|---|
| Target | Host | Clone | Flour | Company | Catalogue # | conc./dilution |
| Human CX3CR1 | Rat | 2A9-1 | APC-Cy7 | Biolegend | 341616 | Flow @1:20 |
| Human CX3CR1 | Rat | 2A9-1 | BV-510 | Biolegend | 341622 | Flow @1:20 |
| Human Granzyme B | Mouse | CLB-GB11 | PERCP | Novus | NBP1-50071PCP | Flow @1:100 |
| Human PD1 | Mouse | EH12.2H7 | APC | Biolegend | 329908 | Flow @1:20 |
| Human PD-1 | Mouse | EH12.2H7 | APC-Cy7 | Biolegend | 329922 | Flow @1:20 |
| Human PD-1 | Mouse | EH12.1 | BV510 | BD Horizon | 563076 | Flow @1:20 |
| Human TCF-1 | Rabbit | C63D9 | Pac-Blue | Cell Signaling | #9066 | Flow @1:50 |
| Human TCF-1 | Rabbit | C63D9 | PE | Cell Signaling | #14456 | Flow @1:50 |
| Human EOMES | Mouse | WD1928 | FITC | eBioscience by | 11-4877-42 | Flow @1:20 |
| | | | | ThermoFisher | | |
| Human TOX | Recombinant | REA473 | PE | Miltenyi Biotec | 130-120-716 | Flow @1:50 |
| Human Tbet | Mouse | 4B10 | APC | Biolegend | 644814 | Flow @1:20 |
| Human HIF-1α | Mouse | Mgc3 | APC | eBioscience by | 17-7528-82 | Flow @1:20 |
| | | | | ThermoFisher | | |
| Human GLUT1 | Mouse | 202915 | FITC | R&D Systems | FAB1418F | Flow @1:20 |
| Human CCR7 | Mouse | 2-L1-A | BV 650 | BD Horizon | 566756 | Flow @1:20 |
| Human CCR7 | Mouse | G043H7 | FITC | Biolegend | 353216 | Flow @1:20 |
| Human CD45RA | Mouse | HI100 | BV 510 | BD Horizon | 563031 | Flow @1:20 |
| Human CD45RA | Mouse | HI100 | APC | eBioscience by | 17-0458-42 | Flow @1:20 |
| | | | | ThermoFisher | | |
| Human CD45RA | Mouse | 5H9 | BUV496 | Biolegend | 741182 | Flow @1:20 |
| Human Ki67 | Mouse | Ki-67 | APC | Biolegend | 350514 | Flow @1:20 |
| Human CD8 | Mouse | RPA-T8 | PE-Cy7 | BD Horizon | 557746 | Flow @1:20 |
| Human CD8 | Mouse | SK1 | BV 510 | Biolegend | 344732 | Flow @1:20 |
| Human CD8 | Mouse | SK1 | APC | Biolegend | 344722 | Flow @1:20 |
| Human CD3 | Mouse | UCHT1 | BV 650 | Biolegend | 300468 | Flow @1:20 |
| Human CD3 | Mouse | UCHT1 | PECy5.5 | Biolegend | 300410 | Flow @1:20 |
| Human CD3 | Mouse | OKT3 | BV 421 | Biolegend | 317344 | Flow @1:20 |
| Human CD11a | Mouse | TS2/4 | PerCP/Cy5.5 | Biolegend | 350614 | Flow @1:20 |
| Human CD11a | Mouse | HI111 | PE-Cy7 | Biolegend | 301220 | Flow @1:20 |
| Human CD11a | Mouse | HI111 | APC-Cy7 | Biolegend | 301236 | Flow @1:20 |
| Human ME1 | Mouse | | None | Santa Cruz | sc-365891 | Western Blot @ 1:1000 |
| Human ME1 | Mouse | | PE | Biotechnology | sc-365891 | Flow @1:50 |
| Human NKG7 | Rabbit | | AF488 | Epigentek | 30-69AA | Flow@1:100 |
| Human CD107a | Mouse | H4A3 | FITC | Biolegend | 328606 | Flow @1:20 |
| Brefeldin A | | | | Biolegend | 420601 | |
| Monensin | | | | Biolegend | 420701 | |
| Mouse IgG | Mouse | Polyclonal | None | | | |
| Rabbit IgG | Rabbit | Polyclonal | None | | | |
| Peroxidase affiniPure Donkey | | Polyclonal | | Jackson | 715-035-151 | Western blot @ 1:5000 |
| anti-mouse IgG (H⁺L) | | | | ImmunoResearch | | |
| Beta-actin | Rabbit | | None | Cell Signaling | #4970S | Western blot @ 1:2000 |

TABLE 1B
Dyes

| Reagent (Fluoro) | Supplier | Catalogue # | Final conc. |
|---|---|---|---|
| Ghost Dye UV 450 (UV405) | Tonbo biosciences | 13-0868-T100 | Flow @1:100 |
| Ghost Dye V 450 (BV450) | Tonbo biosciences | 13-0863-T100 | Flow @1:100 |
| CELLROX ™ Green Flow Kit | ThermoFisher Scientific | C10492 | Flow @500 nM/mL |
| MITOSOX ™ Red | ThermoFisher Scientific | M36008 | Flow @100 nM/mL |
| MITOTRACKER ™ Green FM | ThermoFisher Scientific | M7514 | Flow @100 nM/mL |
| CELLTRACE ™ CFSE dye | ThermoFisher Scientific | C34554 | Flow @ 2.5 μM/mL |
| TMRM, perchlorate | Biotium | 70017 | Flow @0.02 μM |
| TMRM | ThermoFisher Scientific | M20036 | Flow @0.02 μM |

TABLE 1C
CD8⁺ isolation and nucleofection reagents

| Reagent | Supplier | Catalogue # |
|---|---|---|
| Lymphoprep | STEMCELL Technologies | 07851 |
| SepMate Conical tubes | STEMCELL Technologies | 85450 |
| EasySep Human CD8⁺ T cell Isolation Kit | STEMCELL Technologies | Q-263176 |
| P3 Primary Cell 4D-Nucleofector X Kit S | Lonza | V4XP-3032 |
| P3 Primary Cell 4D-Nucleofector X Kit L | Lonza | V4XP-3024 |

TABLE 1D
qPCR reagents

| Reagent | Supplier | Catalogue # |
|---|---|---|
| RLT buffer | Qiagen | 79216 |
| Qiagen RNEASY ® Plus Mini Kit | Qiagen | 74134 |
| SUPERSCRIPT ™ III Reverse Transcriptase | ThermoFisher Scientific | 18080-400 |
| TaqMan Fast Advanced Master Mix | Applied Biosystems | 444455 |

TABLE 1E
Seahorse reagents

| Reagent | Supplier | Catalogue # |
|---|---|---|
| Seahorse XF Cell Mito Stress Test Kit | Agilent | 103015-100 |
| Seahorse XF Glycolysis Stress Test Kit | Agilent | 103020-100 |
| Seahorse XFe96 FluxPak mini | Agilent | 102601-100 |
| Seahorse XF Cell Energy Phenotype Test Kit | Agilent | 103325-100 |
| Seahorse XF RPMI medium pH 7.4, 500 mL | Agilent | 103576-100 |
| Seahorse XF 100 mM pyruvate solution, 50 mL | Agilent | 103578-100 |
| Seahorse XF 200 mM glutamine solution, 50 mL | Agilent | 103579-100 |
| Seahorse XF 1.0M glucose solution, 50 mL | Agilent | 103577-100 |
| Corning Cell-Tak Cell and Tissue Adhesive, 1 mg | Corning | 354240 |

TABLE 1F
Inhibitors

| Reagent | Supplier | Catalogue # | Final conc. | |
|---|---|---|---|---|
| Oligomycin (OCR) | Agilent | 103015-100 | 2.0 | μM |
| FCCP | Agilent | 103015-100 | 1.5 | μM |
| Rot/AA | Agilent | 103015-100 | 1.0 | μM |
| Glucose | Agilent | 103020-100 | 10 | mM |
| Oligomycin (ECAR) | Agilent | 103020-100 | 2.0 | μM |
| 2-DG | Agilent | 103020-100 | 50 | nM |
| JAK1 inhibitor | Biotechne-Tocris | 7783 | 100-1000 | nM |

TABLE 1G
KD and mRNA Overexpression reagents

| Reagent | Company/Catalogue or sequence | Catalogue # |
|---|---|---|
| ME1 siRNA SMARTpool: ON-TARGETplus | Dharmacon | L-009348-00-0020 |
| Control siRNA ON-TARGETplus Non-targeting pool | Dharmacon | D-001810-10-20 |
| ME1 mRNA for human studies | TriLink Biotechnologies | L-7007 |
| Control mRNA | TriLink Biotechnologies | L-7610 |

TABLE 1H
Cell culture reagents

| Reagent | Company | Catalogue # | Final conc/Usage Notes |
|---|---|---|---|
| DMEM | Gibco | 11885-084 | Base media for MCF-7 |
| RPMI | Corning | 10-040-CV | Base media for PC.3 |
| HEPES buffer | Corning | 25-060-CI | Added 10 mL of the 1M solution to |
| | | | 500 mL of all cell culture media |
| FBS | Gibco | 10437-028 | Final concentration of 10% FBS used |
| | | | for all cell culture media |
| FoxP3/TF Fixation/Permeabilization concentrate and diluent | eBioscience by | 00-5521-00 | |
| | ThermoFisher | | |
| Pen/Strep | Cellgro | 30-002-CI | Used 5 mL of the 100× solution in |
| | | | 500 mL of all cell culture media |
| Recombinant human IL-2 | Peprotech | 200-02 | 10 U/ml unless otherwise stated in |
| | | | methods. |
| Recombinant human IL-15 | Peprotech | 200-15 | 10 ng/mL |
| Recombinant human IL-7 | Peprotech | 200-07 | 10 ng/mL |
| Purified NA/LE Mouse Anti-human CD3 (Clone HIT3a) | BD Horizon | 555336 | |
| Purified NA/LE Mouse anti-human CD28 (Clone CD28.2) | BD Horizon | 555725 | |
| Gibco DYNABEADS ™ Human T-Activator CD3/CD28 | Gibco | 11132D | 25 μL/1 × 10⁶ cells |
| IMMUNOCULT ™ Human CD3/CD28 T cell Activator | STEMCELL | 10991 | 25 μL/1 × 10⁶ cells |
| | Technologies | | |
| NADP⁺/NADPH Assay Kit | Abnova | KA1663 | |

### Results

CX3CR1 and Low Mitochondria Membrane Potential Identify Resilient CD8⁺ T Cells in Patients with Advanced Cancers

To reduce the tumor burden in patients with very large or therapy-resistant tumors, patients were treated with a new format of radiation therapy: spatially fractionated radiotherapy (SFRT) (FIG. 2B). SFRT treats some regions of the tumor at very high doses while at the same time sparing both the surrounding normal tissues and other parts of the tumor. SFRT can produce dramatic relief of severe symptoms, cause significant tumor regression, and achieve above average local control rates (Grams et al., Pract Radiat Oncol 11, e339-e347, 2021). The biological mechanisms that are responsible for SFRT are unknown, but the heterogeneous dose distribution may be responsible for modifying tumor-reactive immune response locally and systemically.

CX3CR1⁺ CD8⁺ T cells are less exhausted, and demonstrate high cytotoxic capability in patients with advanced cancers such as melanoma and lung cancers (Wu et al., Nature 579, 274-278, 2020; Yan et al., supra; and Yamauchi et al., Nat Commun 12, 1402, 2021), suggesting that a change of functional CX3CR1⁺ CD8⁺ T cells might reflect an optimal response to a successful SFRT. Given the presence of large tumor burden in patients in which compromised mitochondrial function was linked with T cell exhaustion (Li et al., J Exp Med 219, 2022; Li et al., Immunity 51, 491-507.e497, 2019; Nishida et al., J Immunother Cancer 9, 2021; Ogando et al., J Immunother Cancer 7, 151, 2019; Simula et al., Mol Oncol 16, 188-205, 2022; and Yu et al., Nature Immunology 21, 1540-1551, 2020), the CTL function and mitochondria membrane potential (MMP) of CX3CR1⁺ CD8⁺ T cells was measured. The CTL function of these T cells was measured with CD107a expression ex vivo for a degranulation process involved in cytotoxicity. In patients with advanced lung cancers and sarcomas, CTL function was increased one day after SFRT for CX3CR1⁺ CD8⁺ T cells with low MMP, but not for CX3CR1⁺ CD8⁺ T cells with high MMP (FIG. 2C, ** P<0.01, *P<0.05, n=17). In addition, CX3CR1⁺ CD8⁺ T cells with low MMP had higher CTL function than cells with high MMP one week after SFRT (FIG. 2D). In another cohort of patients with advanced melanoma that was resistant to ICI therapy, CX3CR1⁺ CD8⁺ T cells, but not CX3CR1⁻ CD8⁺ T cells, were found to be enriched with low MMP phenotype in the peripheral blood (FIG. 2E) prior to radiation therapy. This cohort of patients was treated with SBRT to reduce tumor burden. Patients with a higher frequency (>28%) of CX3CR1⁺ CD8⁺ T cells with low MMP at baseline demonstrated a better clinical outcome (survival) in response to SBRT (FIG. 2F, p<0.05, n=20). On the other hand, the frequency of CX3CR1⁺ CD8⁻ T cells with low MMP gradually decreased in responders to ICI therapy as the tumor burden was reduced, but fluctuated in non-responders as the tumor burden was persistent (FIGS. 2G and 2H, respectively). Taken together, these clinical observations suggested that CX3CR1⁺ CD8⁺ T cells with low MMP are present in patients with advanced cancers and have a functional resiliency once the tumor burden is reduced.

### Resilient CD8⁺ T Cells are Highly Cytotoxic and Less Exhausted

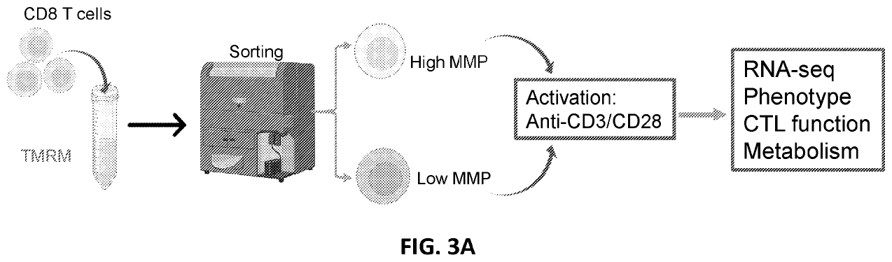

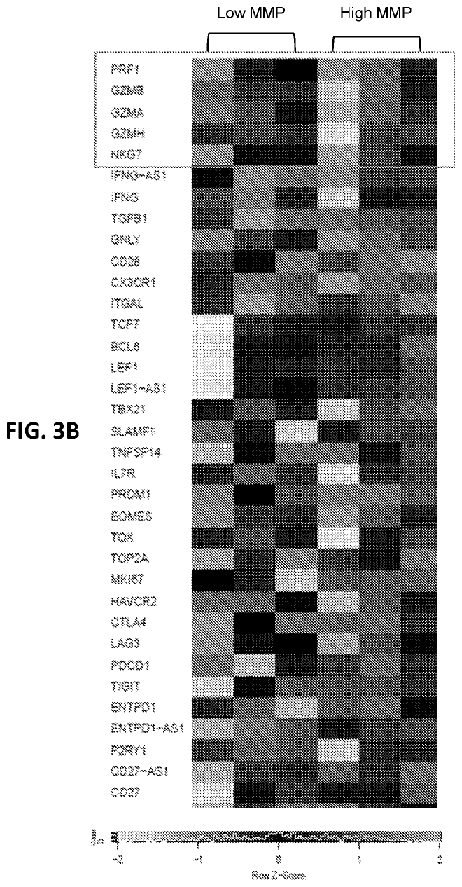

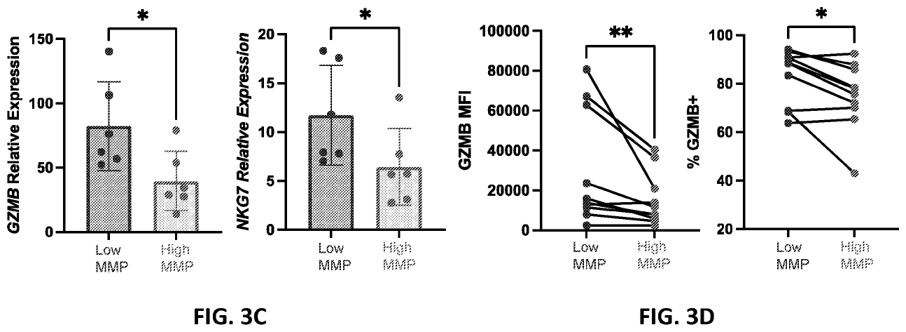

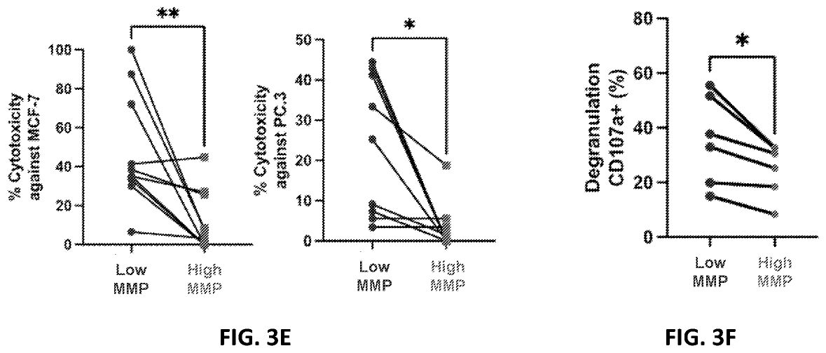

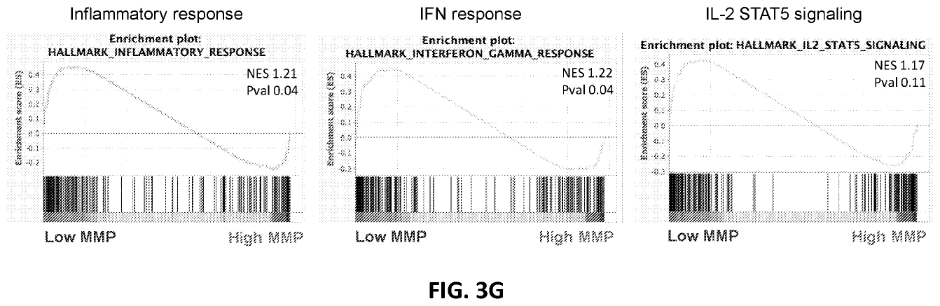

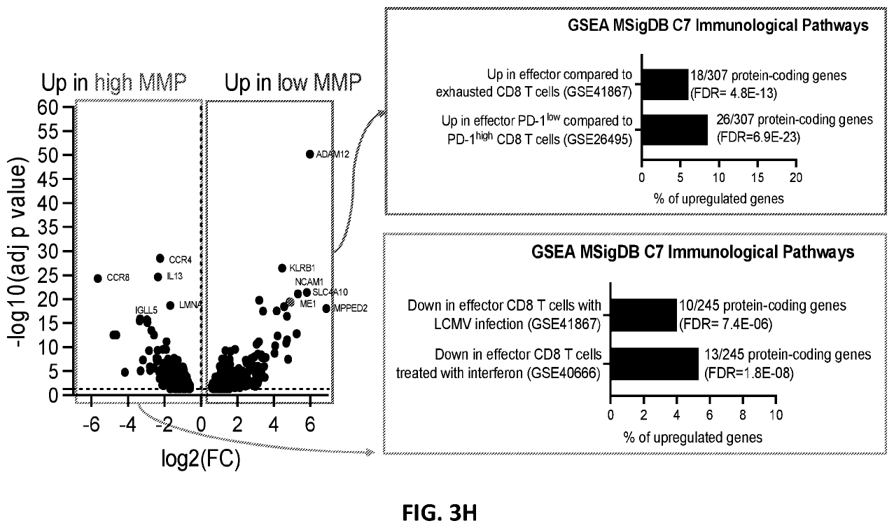

CD8⁺ T cells with low MMP have been reported elsewhere to have increased antitumor activity in preclinical mouse models (Sukumar et al., Cell Metab 23, 63-76, 2016). The data presented herein suggested that CX3CR1⁺ CD8⁺ T cells with low MMP represent a T cell population that is less exhausted and functionally resilient in patients with advanced tumors. To understand how the low MMP feature of CD8⁺ T cells might be linked with their functional state, CD8⁺ T cells were sorted into low or high MMP using TMRM (FIG. 3A) according to the method of Sukumar et al., (supra). First, bulk RNA-seq analysis was performed to compare the transcription differences between CD8⁺ T cells with low and high MMP. These studies demonstrated that CD8⁺ T cells with low MMP expressed more genes that code for cytotoxic effector molecules such as GZMB, PRF1, and NKG7 than CD8⁺ T cells with high MMP (FIG. 3B). A higher expression of granzyme B was then confirmed in CD8⁺ T cells with low MMP compared to CD8⁺ T cells with high MMP using RT-PCR and flow cytometry (FIGS. 3C and 3D). In a T cell-mediated tumor cytotoxicity assay following a brief T cell activation with anti-CD3/CD28, it was found that CD8⁺ T cells with low MMP demonstrated 1.5-fold higher cytolytic activity than to CD8⁺ T cells with high MMP in killing of two tumor cell lines (breast cancer and prostate cancer; FIG. 3E). In addition, significantly higher degranulation was observed in CD8⁺ T cells with low MMP compared to CD8⁺ T cells with high MMP upon T cell activation (FIG. 3F). Further, GSEA and C7 immunological signature analyses demonstrated that CD8⁺ T cells with low MMP were enriched with genes involved in inflammatory responses, including IFN and IL-2/STAT5 pathways (FIG. 3G) and were enriched with genes upregulated in effector/PD-1 low CD8⁺ T cells (FIG. 3H). These results suggested that CD8⁺ T cells with low MMP are endowed with a high cytotoxic capability and are programmed to be responsive to inflammatory signals.

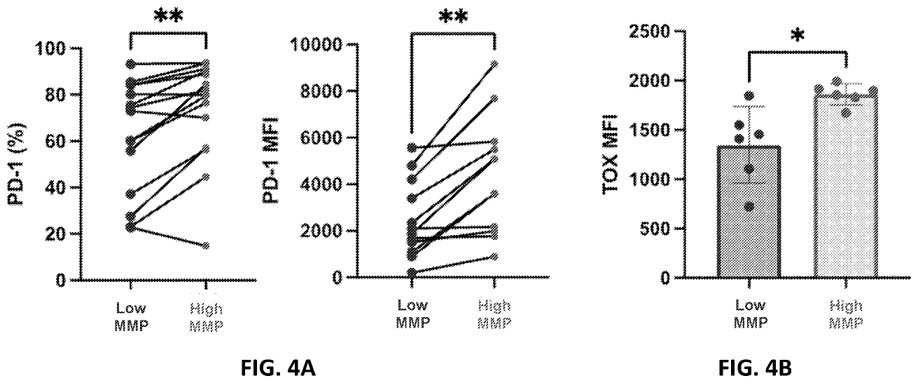

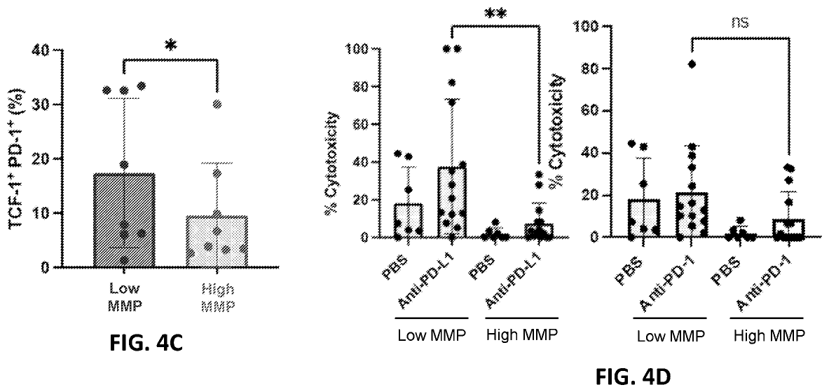

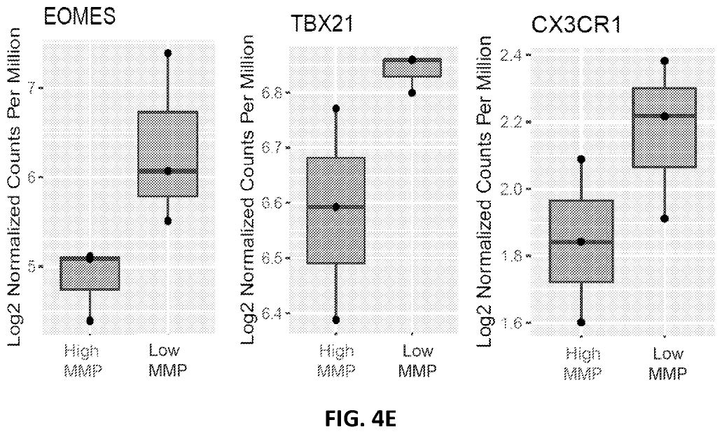

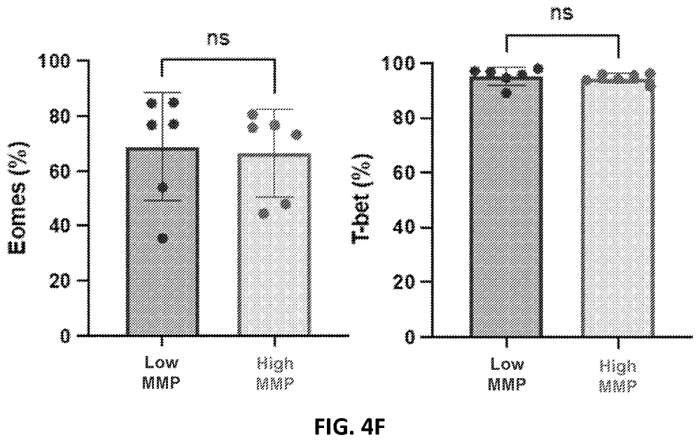

Given the high cytotoxic capability of CD8⁺ T cells with low MMP, studies were conducted to assess whether they would be prone to exhaustion upon activation. Unexpectedly, it was found that CD8⁺ T cells with low MMP had lower expression of exhaustion markers such as PD-1 and TOX (FIGS. 4A and 4B). Interestingly, CD8⁻ T cells with low MMP were enriched with TCF-1⁺PD-1⁺ stem-like cells, which have been reported to be responsive to immunotherapy (FIG. 4C) (Sukumar et al., supra). To confirm this, the cytotoxicity of CD8⁺ T cells with low or high MMP in co-culture with anti-PD-1 or anti-PD-L1 antibody was measured and compared. These studies demonstrated that CD8⁺ T cells with low MMP exhibited increased cytotoxicity in the presence of anti-PD-L1 and anti-PD-1 antibodies as compared to CD8⁺ T cells with high MMP, although only the anti-PD-L1 group reached statistical significance (FIG. 4D). Expression of the transcription factors Eomesodermin (Eomes) and T-bot transcription factor (T-bet) also was compared between CD8⁺ T cells with low or high MMP, as these transcription factors can regulate the expression of effector genes in CD8⁺ T cells (Buggert et al., PLoS Pathog 10, e1004251, 2014; Intlekofer et al., Nat Immunol 6, 1236-1244, 2005; Pearce et al., Science 302, 1041-1043, 2003; and Szabo et al., Cell 100, 655-669, 2000), and the ratio of Eomes/T-bet may determine T cell exhaustion and immune pathology (Llaó-Cid et al., Leukemia, 2021; and Raveney et al., Proc Natl Acad Sci 118, e2021818118, 2021). Although Eomes and T-bet expression at the mRNA level were significantly higher in CD8⁺ T cells with low MMP compared to high MMP cells, the protein levels of Eomes and T-bet were comparable between CD8⁺ T cells with low or high MMP (FIGS. 4E and 4F). These data suggested that although CD8⁻ T cells with low MMP seem to be programmed with the capability of effector cells that could lead to exhaustion (based on high Eomes transcription), their functional state may be regulated by other mechanisms to counterbalance the trend to enter a state of exhaustion.

Resilient CD8⁺ T Cells have Lower Glycolysis and Less ROS

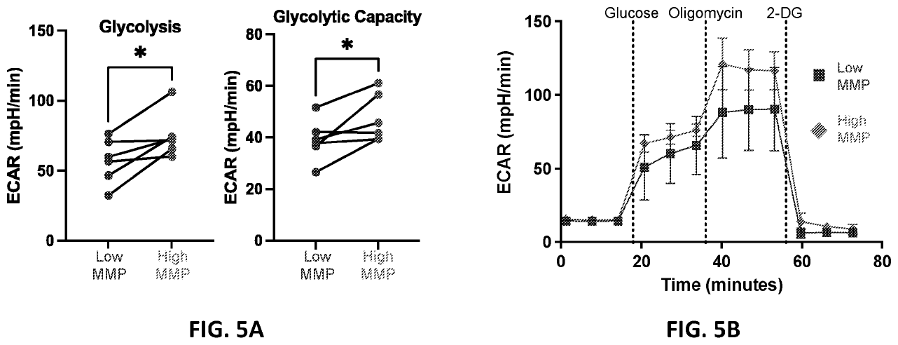

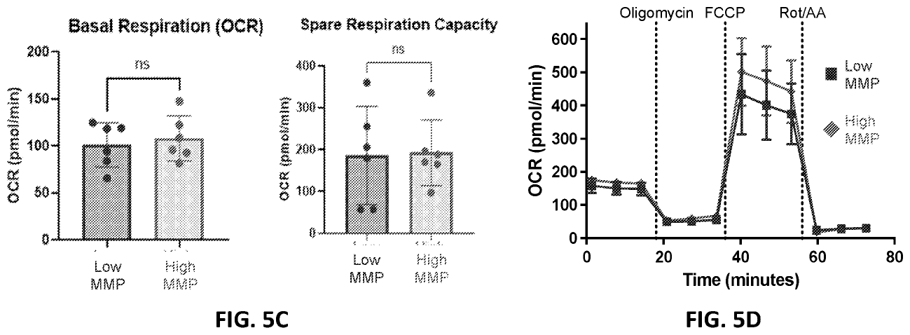

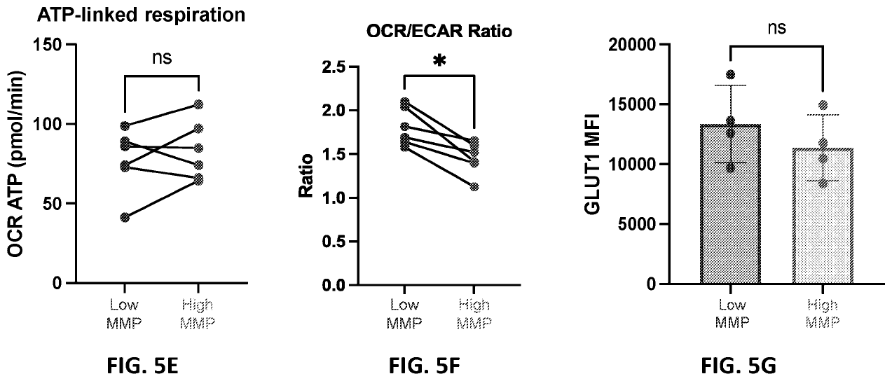

As the levels of MMP of CD8⁺ T cells also reflect the metabolic state of T cells, glycolysis (ECAR) and mitochondria respiration (OCR) were compared for CD8⁺ T cells with low MMP or high MMP. CD8⁺ T cells with low MMP had lower glycolysis and glycolytic capacity than CD8⁺ T cells with high MMP (FIGS. 5A and 5B), while the oxidative phosphorylation (OXPHOS) was comparable between CD8⁺ T cells with low MMP or high MMP (FIGS. 5C and 5D). Given the high cytotoxicity capacity of the CD8⁺ T cells with low MMP, it was unexpected that they exhibited lower glycolysis. However, the mitochondria ATP storage and the spare respiratory capacity are comparable between CD8⁺ T cells with low MMP or high MMP (FIGS. 5D and 5E). Additionally, the OCR/ECAR ratio did not show any significant differences in OXPHOS preference between CD8⁺ T cells with low MMP or high MMP (FIG. 5F). Since GLUT1 levels were comparable between CD8⁺ T cells with low MMP or high MMP (FIG. 5G), the lower glycolysis of CD8⁺ T cells with low MMP may not be due to a lower intake of glucose. These data suggested although CD8⁺ T cells with low MMP do not have high levels of glycolysis, they maintain their metabolic fitness via mitochondrial ATP production.

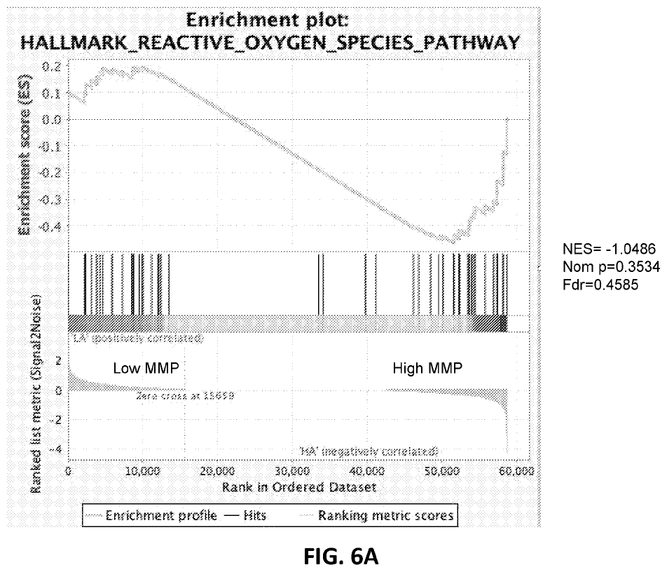

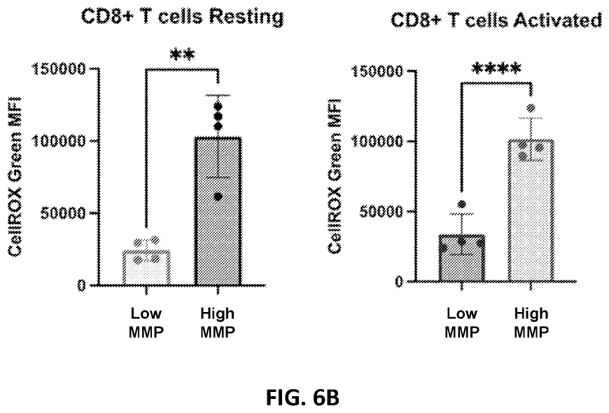

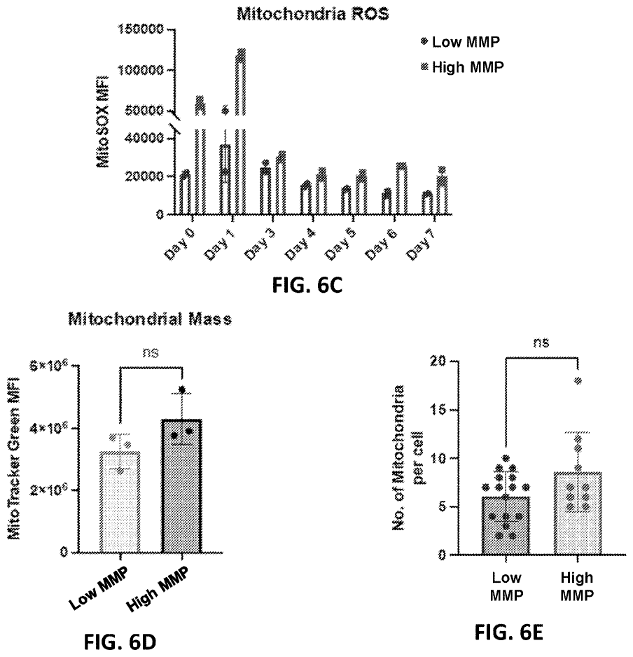

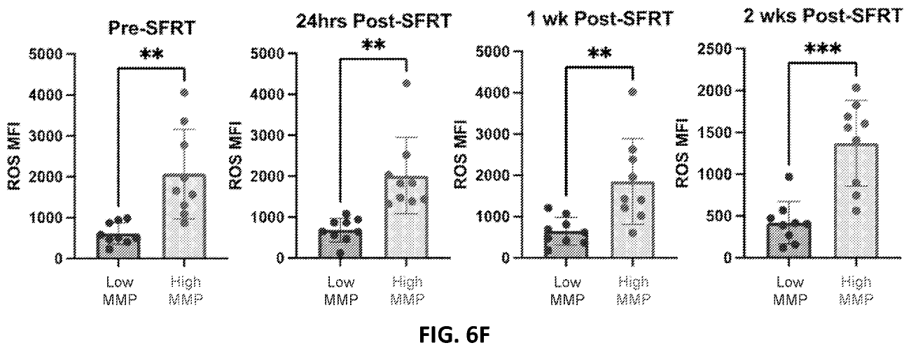

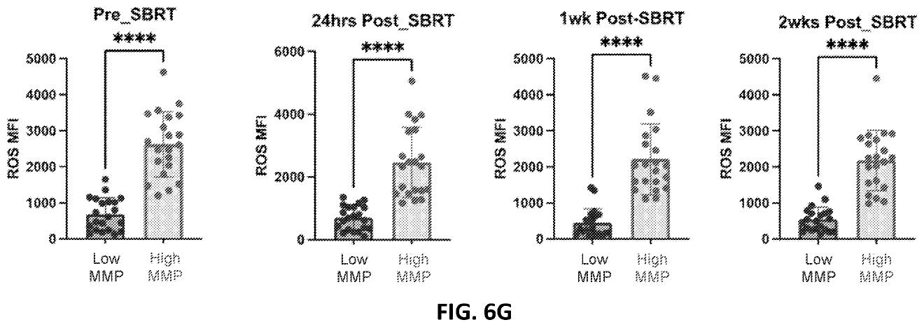

The GSEA analysis revealed that the reactive oxygen species (ROS) pathway was enriched in CD8⁺ T cells with low MMP compared to CD8⁺ T cells with high MMP (FIG. 6A). In further studies, levels of ROS were measured and compared between CD8⁺ T cells with low MMP or high MMP. These studies revealed that both cytosolic ROS and mitochondrial ROS were lower in resting and activated CD8⁺ T cells with low MMP compared to CD8⁺ T cells with high MMP (FIGS. 6B and 6C). The lower levels of ROS were maintained up to 7 days in in vitro culture (FIG. 6C), suggesting that a lower metabolic output in CD8⁺ T cells with low MMP is in place to curtail excessive ROS production. To that end, the mass and numbers of mitochondria were measured and compared between CD8⁺ T cells with low and high MMP. Although there was a trend of lower mass (FIG. 6D) and numbers (FIG. 6E) of mitochondria in CD8⁺ T cells with low MMP, the difference did not reach statistical significance, suggesting that another mechanism may contribute the lower levels of ROS beyond the volume of mitochondria.

Since excessive ROS signals result in not only DNA damage but also exhaustion in T cells (Yu et al., supra; Dan et al., Nature Immunol 21, 287-297, 2020; and Scharping et al., Nature Immunol 22, 205-215, 2021), the ability to maintain a lower ROS levels despite optimal OXPHOS could be a unique feature of CD8⁺ T cells with low MMP that enable them to tolerate harsh environments that would lead to increase ROS in T cells. To test whether this feature of CD8⁺ T cells with low MMP could be retained in T cells in patients with advanced cancers, the levels of ROS measured and compared in CD8⁺ T cells with low or high MMP isolated from patients with advanced lung cancers and sarcoma before and after SFRT. Interestingly, a lower level of ROS was consistently observed in CD8⁺ T cells with low MMP compared to cells with high MMP before and after SFRT (FIG. 6F, n=9). Similar findings were confirmed in another cohort of patients with advanced prostate cancer receiving SBRT (FIG. 6G, n=21). These results suggested that CD8⁺ T cells with low MMP have the capability to maintain lower ROS levels despite the advanced stage.

### ME1 is Upregulated in Resilient CD8⁺ T Cells

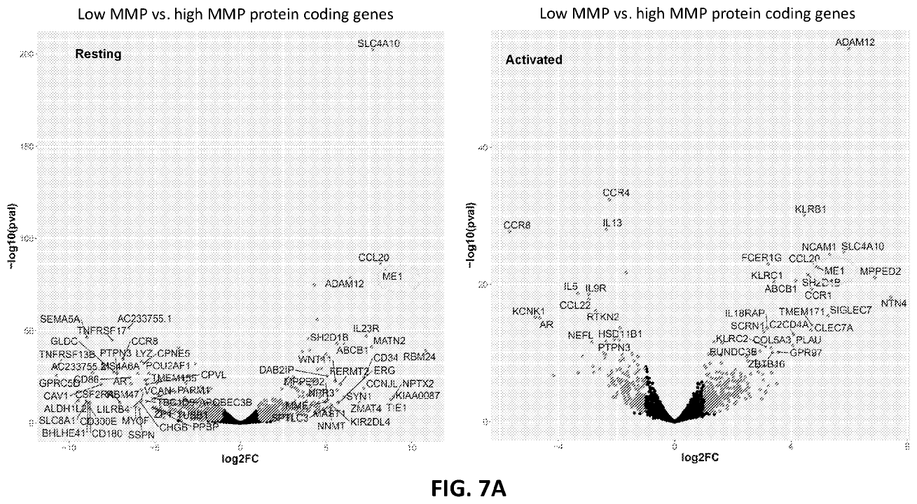

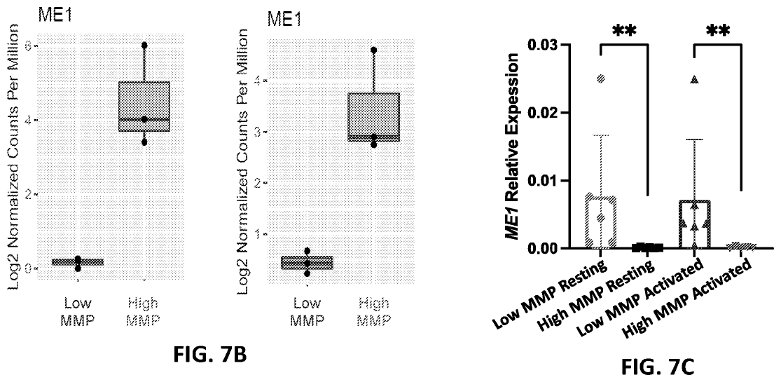

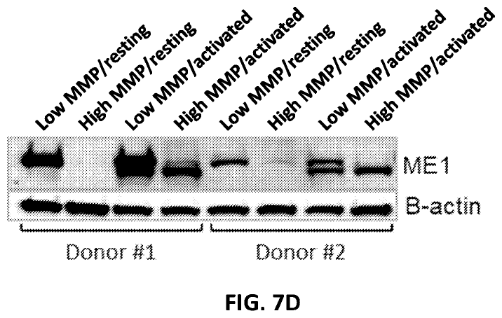

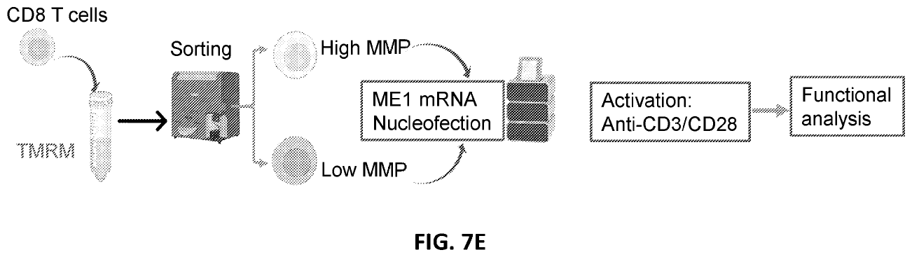

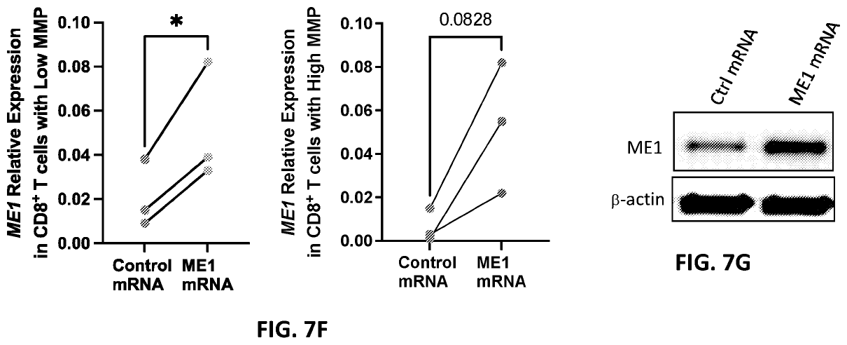

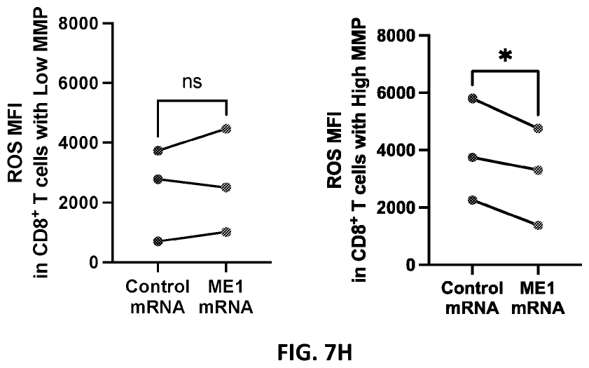

RNA-seq analysis was performed to examine how CD8⁺ T cells with low MMP can maintain their ATP production via OXPHOS while keeping lower ROS levels. ME1 was among the most upregulated genes in the CD8⁺ T cells with low MMP cells in both resting and activated states, compared to CD8⁺ T cells with high MMP (FIGS. 7A and 7B). Of the most upregulated genes, only ME1 has the potential to regulate metabolism. Thus, further studies were focused on ME1, confirming via quantitative RT-PCR and Western blotting that ME1 has higher expression in CD8⁺ T cells with low MMP as compared to CD8⁺ T cells with high MMP, in both resting and activated states (FIGS. 7C and 7D).

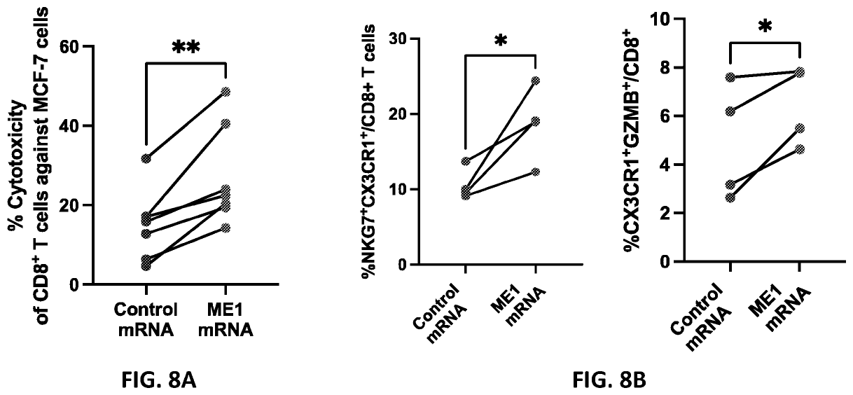

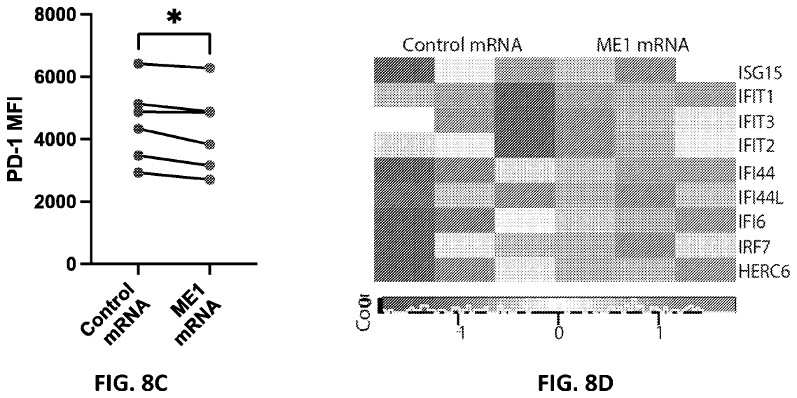

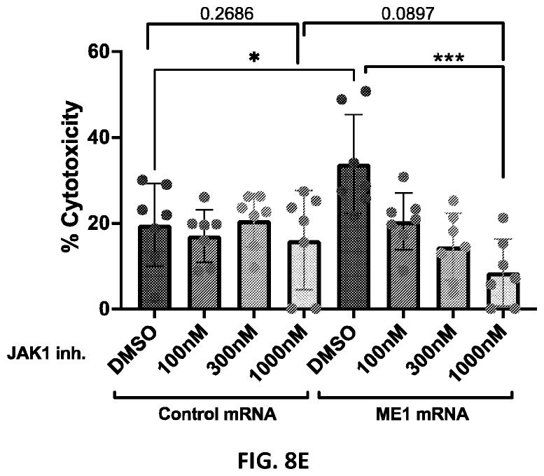

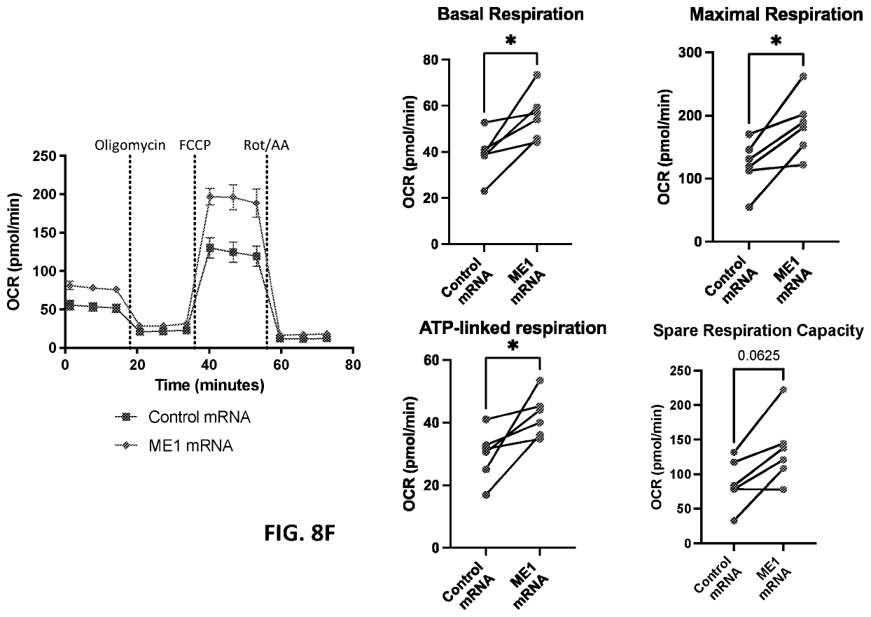

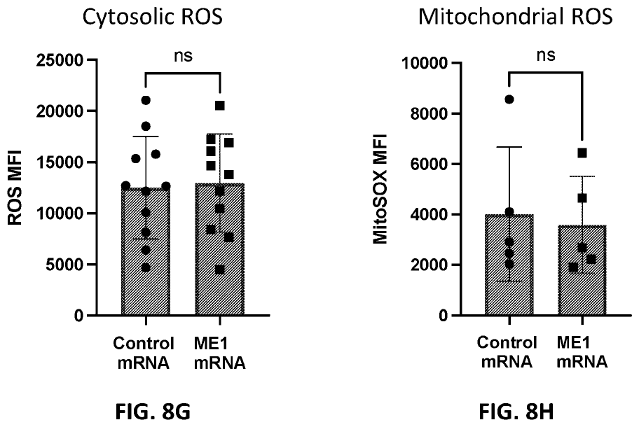

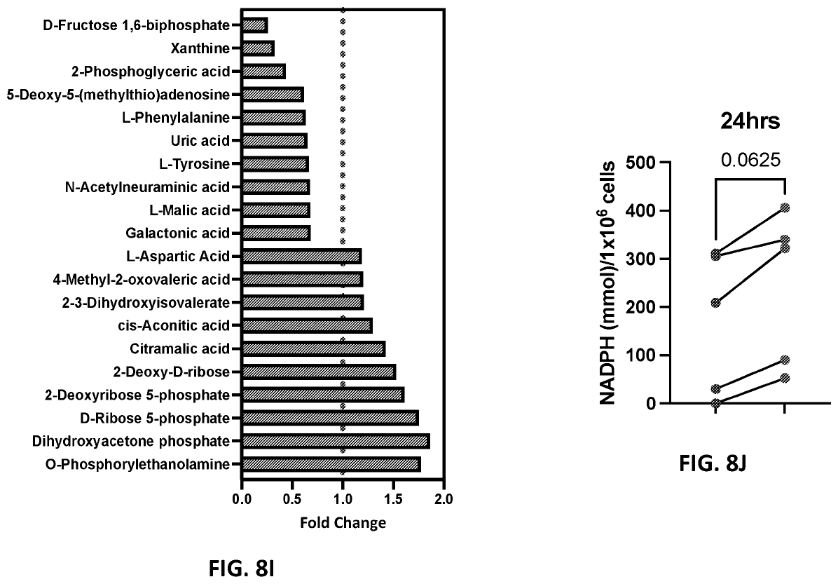

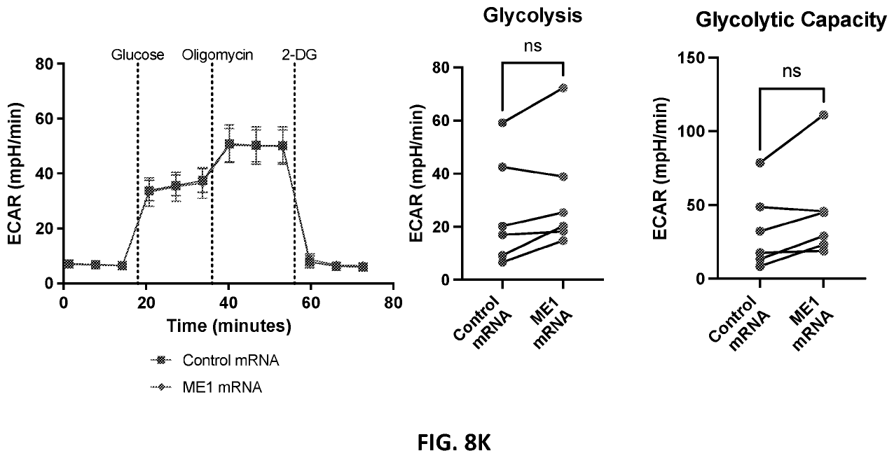

To examine the consequences of ME1 expression in CD8⁺ T cells with low or high MMP, ME1 was overexpressed in CD8⁺ T cells using nucleofection of ME1 mRNA followed by functional analysis (FIG. 7E). The overexpression of ME1 was confirmed by RT-PCR in CD8⁺ T cells with low or high MMP and Western blotting in CD8⁺ T cells (FIGS. 7F and 7G). Further studies showed that ME1 overexpression reduced ROS levels in CD8⁺ T cells with high MMP but not in CD8⁺ T cells with low MMP (FIG. 7H), likely since CD8⁺ T cells with low MMP already had less ROS (FIGS. 6B and 6C) so ME1 was not able to further reduce ROS in those cells. To test whether overexpression of ME1 would affect the cytotoxicity of CD8⁺ T cells, T cell-mediated cytotoxicity was measured and compared between CD8⁺ T cells transfected with control or ME1 mRNA. Interestingly, these studies revealed that ME1 overexpression significantly increased the cytotoxicity of CD8⁻ T cells in killing tumor cells in vitro (FIG. 8A). According, ME1 overexpression significantly increased the frequency of CX3CR1⁺NKG7⁻ and the frequency of CX3CR1⁺GZMB⁺ among CD8⁺ T cells after ex vivo activation (FIG. 8B), but decreased PD-1 levels (FIG. 8C). Genes belonging to the type I IFN-stimulated gene (ISGs) family were among the genes that were significantly upregulated in ME1 mRNA-transfected CD8⁻ T cells (FIG. 8D). To determine whether the signals of type I interferon signaling pathway would affect ME1-enhanced cytotoxicity, a JAK1 inhibitor (Upadacitinib) was used during activation of T cells transfected with ME1 or control mRNA. Interestingly, the JAK1 inhibitor demonstrated a dose-dependent effect in inhibition of cytotoxicity of CD8⁺ T cells transfected with ME1 mRNA, but not in T cells transfected with control mRNA (FIG. 8E). These data suggested that ME1 overexpression promoted T cell cytotoxicity partially through reprograming the type I interferon pathway.

To examine the role of ME1 overexpression in CD8⁺ T cells metabolic fitness, CD8⁺ T cell metabolism was evaluated following ME1 overexpression compared to control mRNA. ME1 overexpression was found to significantly increase basal and maximal mitochondrial respiration as well as a higher ATP-linked respiration in CD8⁺ T cells, along with an augmented spare respiratory capacity (SRC) (FIG. 8F). However, ME1 overexpression did not increase the cytosolic or mitochondrial levels of ROS among CD8⁺ T cells (FIGS. 8G and 8H). Central Carbon Metabolism (CCM) analysis was performed to investigate how ME1 regulates the energy production and redox state. ME1 overexpression significantly reduced D-fructose 1,6-bisphosphate and 2-phosphoglyceric acid, which are intermediate metabolites in the glycolytic pathway. ME1 overexpression also increased ribose 5-phosphate (R5P), a key metabolite in the pentose phosphate pathway (PPP), a main pathway that produces NADPH (FIG. 8I). Accordingly, ME1-overexpressing CD8⁺ T cells had higher NADPH concentrations at 24 hours post activation compared to control cells, but not after 48 hours (FIG. 8J). However, Seahorse analysis did not identify a significant change in glycolysis of ME1 overexpressing CD8⁺ T cells (FIG. 8K). Collectively, these data suggested that ME1 overexpression selectively targeted pentose phosphate metabolic pathway to balance excessive ROS from an increased mitochondrial respiration in CD8⁺ T cells.

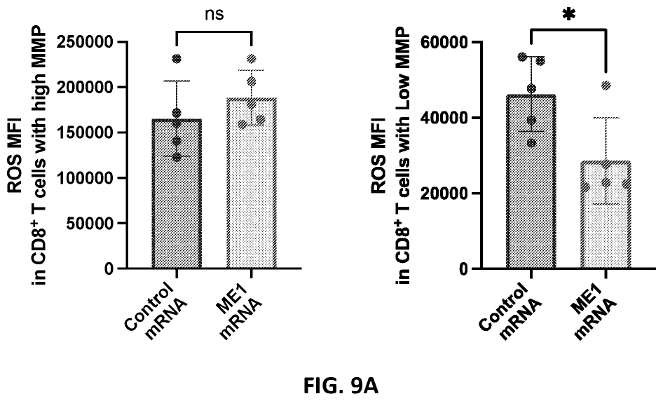

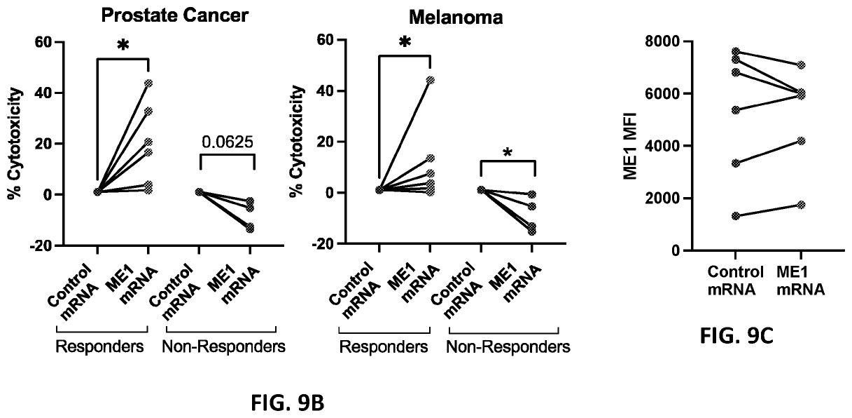

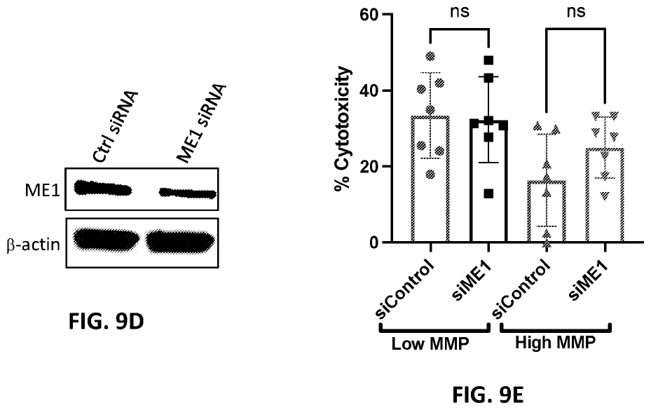

To determine whether ME1 has a therapeutic potential in improving CTL function of PBMC of patients with advanced cancers, ME1 mRNA was introduced into PBMCs isolated from patients with advanced melanoma and prostate cancer. As in CD8⁺ T cells isolated from healthy donors, ME1 overexpression reduced the levels of ROS in activated CD8⁺ T cells. In contrast to healthy donors (FIG. 7H), however, ME1 overexpression reduced ROS in CD8⁺ T cells with low MMP but not in CD8⁺ T cells with high MMP (FIG. 9A). It is possible that CD8⁺ T cells with low MMP in patients with advanced cancers accumulated more ROS that became sensitive to ME1-mediated regulation. Given the potential of ME1 overexpression in promoting cytotoxic CD8⁺ T cells, further studies were conducted to determine whether ME1 could improve CTL function of peripheral lymphocytes in patients with advanced cancers that did not respond to initial ICI therapy. ME1 mRNA was introduced into peripheral lymphocytes isolated from patients with advanced melanoma or prostate cancer, revealing that ME1 overexpression increased the tumor killing ability of peripheral lymphocytes in about 50% of patients with advanced cancers (FIG. 9B). ME1 expression in CD8⁺ T cells that received ME1 mRNA transduction was measured, showing that three of six patients had increased ME1 protein expression, while the other three patients had reduced ME1 protein expression (FIG. 9C). Of note, the three patients with reduced ME1 expression had higher basal levels of ME1 than the three patients who had increased ME1 expression after ME1 mRNA transfection. Thus, the difference of ME1 expression may explain why some patients were responsive to ME1 transfection in the context of increased cytotoxicity.

ME1 was knocked down using siRNA in CD8⁺ T cells in order to determine whether ME1 is necessary for CTL function. However, the ME1 siRNA did not significantly change the cytotoxicity of CD8⁺ T cells with low or high MMP (FIGS. 9D and 9E), demonstrating that although ME1 is not necessary for CTL function, ME1 can be a sufficient factor in induction of highly cytotoxic effector T cells.

Taken together, the studies described herein revealed a new mechanism by which highly cytotoxic function can be maintained in resilient T cells in patients with advanced cancers, providing an avenue to improve the combination of cancer immunotherapy and radiation therapy for patients with advanced diseases that are refractory to current therapy. Leveraging this knowledge of T cell resiliency may improve the efficacy of ICI therapy, CAR-T cell, and/or TCR-T cell therapy to control metastatic diseases that require robust systemic anti-tumor immunity.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for increasing a level of malic enzyme 1 (ME1) in a cell, said method comprising:
introducing into said cell a nucleic acid encoding ME1, and
incubating said cell such that said nucleic acid is expressed, thereby increasing the level of ME1 in said cell.

2. The method of claim 1, wherein said cell is a T cell.

3-7. (canceled)

8. The method of claim 1, wherein the nucleic acid encoding ME1 comprises the nucleotide sequence set forth in SEQ ID NO:8, or having a nucleotide sequence with at least 90% identity to the nucleotide sequence set forth in SEQ ID NO:8.

9. A method for treating a mammal, said method comprising administering to said mammal a composition comprising cells that comprise an exogenous nucleic acid encoding ME1, such that said cells have an elevated level of ME1.

10. The method of claim 9, wherein said mammal is a human.

11. The method of claim 10, wherein said human has cancer.

12-13. (canceled)

14. The method of claim 9, wherein are said cells are T cells.

15-19. (canceled)

20. The method of claim 9, wherein the nucleic acid encoding ME1 comprises the nucleotide sequence set forth in SEQ ID NO:8, or having a nucleotide sequence with at least 90% identity to the nucleotide sequence set forth in SEQ ID NO:8.

21. A composition comprising PBMCs that comprise an exogenous nucleic acid encoding ME1.

22-26. (canceled)

27. A method for increasing a level of a polypeptide having malic enzyme 1 (ME1) activity in a cell, wherein said method comprises:
(a) introducing into said cell a nucleic acid encoding said polypeptide, and
(b) incubating said cell such that said nucleic acid is expressed, thereby increasing the level of said polypeptide in said cell.

28-30. (canceled)

31. The method of claim 27, wherein said cell is a T cell.

32-34. (canceled)

35. A method for treating a mammal, wherein said method comprises administering to said mammal a composition comprising cells comprising an exogenous nucleic acid that encodes a polypeptide having ME1 activity, wherein said cells have an elevated level of said polypeptide.

36-38. (canceled)

39. The method of claim 35, wherein said mammal is a human.

40. The method of claim 39, wherein said human has cancer.

41. The method of claim 40, wherein said cancer is lung cancer, breast cancer, prostate cancer, colorectal cancer, gastric cancer, pancreatic cancer, melanoma, sarcoma, or ovarian cancer.

42. (canceled)

43. The method of claim 35, wherein are said cells are T cells.

44. The method of claim 43, wherein said T cells are CTLs.

45. The method of claim 44, wherein said CTLs are CX3CR1⁺ CTLs.

46. The method of claim 43, wherein said T cells are CAR-T cells or TCR-T cells.

47. The method of claim 35, wherein said cells were obtained from said mammal and transfected with said nucleic acid.

48. (canceled)
